(19) United States
(12) Patent Application Publication
(10) Pub. No.: US 2025/0276054 A1
(43) Pub. Date: Sep. 4, 2025

(54) AVIAN INFLUENZA NANOPARTICLE IMMUNOGENIC COMPOSITIONS

(71) Applicant: Novavax, Inc., Gaithersburg, MD (US)

(72) Inventors: Gale SMITH, Gaithersburg, MD (US); Nita PATEL, Gaithersburg, MD (US); Asma REHMAN, Gaithersburg, MD (US); Jessica F. TROST, Gaithersburg, MD (US); Rhonda FLORES, Gaithersburg, MD (US)

(21) Appl. No.: 19/070,329

(22) Filed: Mar. 4, 2025

Related U.S. Application Data
(60) Provisional application No. 63/561,067, filed on Mar. 4, 2024.
(60) Provisional application No. 63/719,985, filed on Nov. 13, 2024.

(51) Int. Cl. A61K 39/145; A61K 39/00; A61P 37/04

(52) U.S. Cl. CPC A61K 39/145; A61P 37/04; A61K 2039/54; A61K 2039/543; A61K 2039/55555

(57) ABSTRACT

An influenza vaccine nanoparticle includes a recombinant avian influenza hemagglutinin (HA) glycoprotein, where the HA glycoprotein is derived from Type A influenza, subtype A (H5N1) and a Matrix-M adjuvant. The HA glycoprotein has a hydrophobic C-terminus associated with a component of the Matrix-M adjuvant, the component of the Matrix-M being a Matrix-A particle or a Matrix-C particle. In another embodiment, the invention is directed to a vaccine composition having a recombinant glycoprotein antigen with a C-terminus, and a Matrix-M adjuvant. The C-terminus of the glycoprotein antigen is hydrophobic and is associated with a component of the Matrix-M adjuvant.

### RELATED APPLICATIONS

The present application claims priority to U.S. Provisional Patent Application No. 63/561,067, filed on Mar. 4, 2024 and to U.S. Provisional Patent Application No. 63/719,985, filed on Nov. 13, 2024, the contents of which are incorporated herein in their entirety by reference.

### REFERENCE TO AN ELECTRONIC SEQUENCE LISTING

This application contains a Sequence Listing which has been submitted electronically in XML format and is hereby incorporated by reference in its entirety. Said XML copy, created on Feb. 28, 2025 is named 1450_018US1_Sequence_Listing_02_28_2025.xml and is 9,297 bytes in size.

### FIELD OF THE INVENTION

The present invention generally relates to immunogenic compositions for intramuscular or intranasal delivery. The immunogenic compositions contain nanoparticles comprising viral glycoproteins and saponin-based Matrix-M adjuvant. Exemplary viral glycoproteins include Avian influenza virus hemagglutinin (HA) strains. The nanoparticles improve innate and adaptive immunity because of increased antigen density. The invention also provides methods for producing the immunogenic compositions and methods of stimulating immune responses with the compositions. The invention also provides methods of administering the immunogenic compositions.

### BACKGROUND OF THE INVENTION

The emergence of highly pathogenic avian influenza (HPAI) A (H5N1) viruses makes the development of safe, effective, and ideally single-dose vaccines a public health priority. The first case of HPAI A (H5N1) transmission to a human was reported in 1997 in Hong Kong. Since then, the World Health Organization (WHO) has documented over 800 cases of A (H5N1) infection in humans with a fatality rate of over 50%. Recent outbreaks of A (H5N1) influenza have reached panzootic levels with infections in wild birds, poultry, and around 50 mammalian species (mink, sea lions, and others), increasing the pandemic public health risk. A recent spike in A (H5N1) infections in the United States has occurred since early 2024, including approximately dairy cow herds, flocks of birds (commercial and backyard), and human cases (all linked to exposure to infected dairy cows or poultry. Although the current risk of a pandemic from A (H5N1) clade 2.3.4.4b remains low for humans, ongoing transmission events between wild birds, domesticated poultry/cattle, and mammals in close contact with humans will increase the possibility of human-to-human transmissibility with pandemic potential.

Avian influenza virus “A (H5N1)” is characterized by hemagglutinin (HA) with a polybasic cleavage site as seen in other viral glycoproteins, such as SARS-COV-2 envelope spike protein. The A (H5N1) clade 2.3.4.4b was introduced in 2020 in the U.S. causing significant losses in the poultry industry, infection of mammals and several humans in close contact with infected birds. The WHO reported A (H5N1) case fatality rates of 53%. In 2023, the WHO recommended A (H5N1) clade 2.3.4.4b as a candidate vaccine strain.

There is a need in the art for the development of effective immunogenic compositions to vaccinate populations against avian influenza “A (H5N1”).

### SUMMARY OF THE INVENTION

In one embodiment, the invention is directed to an influenza vaccine nanoparticle formed of a recombinant avian influenza hemagglutinin (HA) glycoprotein, wherein the HA glycoprotein is derived from Type A influenza, subtype A (H5N1), and of a Matrix-M adjuvant.

In some aspects, the HA glycoprotein has a hydrophobic C-terminus associated with a component of the Matrix-M adjuvant.

In some aspects, the component of the Matrix-M adjuvant comprises a Matrix-A particle and a Matrix-C particle, and the hydrophobic C-terminus of the HA glycoprotein is associated with the Matrix-A particle or the Matrix-C particle.

In some aspects, the HA glycoprotein has a sequence with at least 90% identity to, at least 95% identity to, at least 97% identity to, at least 98% identity to, at least 99% identity to, or 100% identity to any of SEQ ID NOs. 1-3.

In some aspects, the HA glycoprotein comprises a cytoplasmic tail (CT), a transmembrane (TM) domain and an ectodomain region, and the CT and TM domain comprise 100% identity respectively to CT and TM domains of any of SEQ ID Nos. 1-3, while the ectodomain region has a sequence with at least 90% identity to, at least 95% identity to, at least 97% identity to, at least 98% identity to, at least 99% identity to, or 100% identity to an ectodomain region of any of SEQ ID Nos. 1-3.

In some aspects, the HA: Matrix-M adjuvant mass ratio is about 15:50 or about 15:75 or about 60:50 or about 60:75 or about 180:50 or about 180:75.

Another embodiment includes an immunogenic influenza composition comprising the vaccine nanoparticle and a pharmaceutically acceptable buffer.

In some aspects, the Matrix-M adjuvant is present in the immunogenic influenza composition at about 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.

In some aspects, the HA glycoprotein is present in the immunogenic influenza composition at 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.

Another embodiment is directed to method of preparing a vaccine nanoparticle that includes extracting the HA glycoprotein from a host cell using a first detergent, exchanging the first detergent with a second detergent to form a purified detergent-core nanoparticle comprising the HA glycoprotein and the second detergent, and incubating Matrix-M adjuvant with the purified detergent-core nanoparticle to form a Matrix component nanoparticle (MNP) comprising a Matrix-M component and the HA glycoprotein.

In some aspects, the Matrix-M adjuvant and the purified detergent-core nanoparticle are incubated with an initial respective molar ratio of 1:40.

In some aspects, the Matrix-M adjuvant is incubated with the purified detergent-core nanoparticle for a time of at least four hours.

In some aspects, the HA glycoprotein is expressed in the host cell using a baculovirus.

In some aspects, the host cell is an insect Sf9 cell or Sf22 cell.

In some aspects, the second detergent is PS80.

Another embodiment is directed to a method of stimulating an immune response against influenza comprising administering the immunogenic influenza composition to a subject.

In some aspects, administering the immunogenic influenza composition is performed after the subject has been administered a seasonal influenza vaccine of embodiment.

In some aspects, the composition is administered intramuscularly.

In some aspects, the composition is administered intranasally.

In some aspects, administering the immunogenic influenza composition comprises administering about 60 μg of HA glycoprotein.

In some aspects, administering the immunogenic influenza composition comprises administering about 75 μg of Matrix-M adjuvant.

Another embodiment is directed to a prefilled syringe containing the immunogenic influenza composition.

Another embodiment is directed to a prefilled intranasal delivery device containing the immunogenic influenza composition.

Another embodiment is directed to a vaccine composition comprising a recombinant glycoprotein antigen, the recombinant glycoprotein antigen having an N-terminal and a C-terminal, a Matrix-M adjuvant, wherein the C-terminal of the glycoprotein antigen is hydrophobic and is associated with a component of the Matrix-M adjuvant; and a pharmaceutically acceptable carrier.

In some aspects, the component of the Matrix-M adjuvant is a Matrix-A particle or a Matrix-C particle, and the C-terminus of the recombinant glycoprotein antigen is associated with the Matrix-A particle or the Matrix-C particle.

In some aspects, the recombinant glycoprotein is derived from an influenza hemagglutinin.

In some aspects, the influenza hemagglutinin is derived from type A influenza, subtype H5.

In some aspects, wherein Matrix-M adjuvant comprises Matrix-A particles and Matrix-C particles mixed at a weight ratio of 85:15.

In some aspects, the Matrix-M adjuvant is present at about 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.

In some aspects, the recombinant antigen is present at 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention may be more completely understood in consideration of the following detailed description of various embodiments of the invention in connection with the accompanying drawings.

FIGS. 1A-1D show information related to the characterization of the recombinant influenza A/American Wigeon/South Carolina/22-000345-001/2021 (A/AW/SC/2021) hemagglutinin (HA) antigen. FIG. 1A presents a linear diagram of the full-length HA from A (H5N1) clade 2.3.4.4b A/AW/SC/2021 with the polybasic cleavage site deletion AKRRK. The diagram shows the HA construct from the N- to the C-terminus including the globular head region containing the signal peptide (SP), and the stalk/stem region containing the fusion peptide (FP), transmembrane domain (TMD), and cytoplasmic tail (CT) structural elements. FIG. 1B shows a reduced SDS-PAGE gel with Coomassie blue staining of purified recombinant A (H5N1) A/AW/SC/2021 HA (left) and western blot (right) using an anti-H5N1 HA primary antibody to confirm the identity of the main protein product. FIG. 1C shows representative electron micrographs of negative stained A (H5N1) A/AW/SC/2021 HA proteins as pre-fusion HA-trimers anchored in detergent micelles. Negative staining TEM-2D class average images of A/AW/SC/2021 HA nanoparticles shown with one or two HA-trimers associated with detergent micelle. FIG. 1D shows a representation of a 3D-reconstructed model of A/AW/SC/2021 HA from cryoEM map at 4.2 Å (left). A molecular model for A/AW/SC/2021 HA was generated using PDB ID 6HJR as a template and superimposed into EM density to represent an intact full-length A/AW/SC/2021 HA nanoparticle (front view on the left and top view on the right).

FIGS. 2A-2F show structural and biophysical characterization of A (H5N1) A/AW/SC/2021 HA-Matrix component nanoparticle (MNP) formation. FIG. 2A shows a NS-TEM 3D reconstruction of an A (H5N1) A/AW/SC/2021 HA detergent-core nanoparticle. FIG. 2B shows a NS-TEM 3D reconstruction of a Matrix-M component nanoparticle (Matrix-A or Matrix-C nanoparticle) having a 45-50 nm diameter, at a resolution of 9.8 Angstroms. FIG. 2C shows results of high-performance size-exclusion chromatography (HP-SEC) analysis of a mixture of 30 μg/mL A/AW/SC/2021 HA nanoparticles with 150 μg/mL Matrix-M at time 0 and after incubation at room temperature for 2-12 h (hr), showing the percentage of H5-MNP formation as % bound across time. Curve 202=Ha at t=0. Curve 204=Matrix at T=0. Curve 206=MNP at 2 hours (83.7% of HA being bound to Matrix). Curve 208=MNP at 4 hours (90.3% bound). Curve 210=MNP at 6 hours (93.3% bound). Curve 212=MNP at 8 hours (93.8% bound). Curve 214=MNP at 12 hours (95.5% bound). FIG. 2D shows a NS-TEM landscape characterizing H5-MNPs mixed at an HA: Matrix-M molar ratio of approximately 40:1 (corresponding to 120 μg/mL: 75 μg/mL concentration ratio by mass), at time t=0. FIG. 2E shows a NS-TEM landscape characterizing H5-MNPs mixed at an HA: Matrix-M molar ratio of approximately 40:1, at time t=24 hours. FIG. 2F shows a 3D model of an H5-MNP, with Matrix-M shown in grayscale and H5 trimers (PDB 6HJR shown in dark) docked to the Matrix-M vertex to mimic the MNP formation from 2D classification.

FIGS. 3A-3J illustrate that A/AW/SC/2021 (A/AW) H5-MNP vaccine elicits seroconverting antibody- and cell-mediated immune responses in mise when administered IM or IN. FIG. 3A shows the study protocol in which mice (n=3-10/group) were immunized on Study Days 0 and 21 with a two-dose series of 1 μg A/AW/SC/2021 HA with 5 μg Matrix-M adjuvant intramuscularly (IM), 1 μg A/AW/SC/2021 HA with 5 μg Matrix-M adjuvant intranasally (IN), or 10 μg A/AW/SC/2021 HA with 5 μg Matrix-M adjuvant (IN). In FIG. 3B hemagglutinin inhibiting (HAI) antibody titers against A/AW/SC/2021 were determined in serum collected on Study Day 34 or 35 (2 weeks after completion of the primary series). In FIG. 3C HAI pseudovirus neutralizing titers against A/AW/SC/2021 were determined in serum collected on Study Day 34 or 35 (2 weeks after completion of the primary series). In FIG. 3D, Anti-A/AW IgA and IgG titers were determined in bronchoalveolar lavage (BAL) fluid on Study Day 34 or 35. In FIG. 3E, polyfunctional Triple Th1⁺ cytokine ((IFN-γ, IL-2, TNF-α) CD4⁺ T cell responses were evaluated in spleen tissue collected on Study Day 34/35 from groups that received the intranasal H5-MNP vaccine. In FIG. 3F, polyfunctional Triple Th1⁺ cytokine ((IFN-γ, IL-2, TNF-α) CD4⁺ T cell responses were evaluated in lung tissue collected on Study Day 34/35 from groups that received the intranasal H5-MNP vaccine. FIG. 3G shows Th1 cytokine (IFN-γ, IL-2, TNF-α) and Th2 cytokine (IL-4) responses analyzed in mouse spleen effector CD4⁺ T cells on Study Day 34/35 (n=3-6 per group). Asterisks indicate differences from the placebo-treated group as no differences among vaccinated groups were statistically significant. FIG. 3H shows Th1 cytokine (IFN-γ, IL-2, TNF-α) and Th2 cytokine (IL-4) responses in mouse lung resident CD4⁺ T cells on Study Day 34/35 (n=3-6 per group). FIG. 3I shows individual cytokine responses (IFN-γ, IL-2, TNF-α) in the mouse spleen effector CD8⁺ cells at day 34/35, for placebo, 1 μg IM, 1 μg IN and 10 μg IN. FIG. 3J compares individual cytokine (IFN-γ, IL-2, TNF-α) in murine lung resident CD8⁺ T cells at day 34/35 for IN doses of 1 μg and 10 μg. Symbols represent individual data points, bars represent group geometric mean titers, error bars represent 95% confidence intervals, and the horizontal dashed line represents the assay limit of quantification (LOQ) or limit of detection (LOD) or the seroconverting threshold titer of 1:40. Differences between groups were evaluated by Kruskal-Wallis multiple comparisons test or Mann-Whitney U Test. *p≤0.05; **p≤0.005; ***p<0.0005. Only statistically significant differences are indicated in the figure.

FIGS. 4A-4J show that A/AW/SC/2021 (A/AW) H5-MNP vaccine elicits seroconverting antibody- and cell-meditated immune responses when administered IM or IN in non-human primates (NHPs) primed with quadrivalent nanoparticle seasonal influenza vaccine. FIG. 4A illustrates the study protocol, Rhesus macaques (NHPs) were immunized by a two-dose quadrivalent nanoparticle seasonal influenza vaccine (qNIV; 60 μg HA per strain) with 75 μg Matrix-M adjuvant (IM) on Study Days 0 and 21 (n=10). NHPs (n=5/group) were then administered two sequential booster doses of H5-MNP vaccine (either two IM 60 μg doses of HA or one IN dose of 240 μg HA followed by one IN dose of 60 μg HA; all with 75 μg Matrix-M adjuvant) on Study Days 83 and 153. FIG. 4B shows hemagglutinin inhibiting (HAI) antibody titers against A/AW/SC/2021 evaluated in sera collected on Study Days indicated on the X axis. FIG. 4C shows HAI pseudovirus neutralizing antibody titers against A/AW/SC/2021 evaluated in sera collected on Study Days indicated on the X axis. FIG. 4D shows triple Th1⁺ CD4⁺ T cell responses evaluated in PBMCs collected on Study Days indicated on the X axis. Symbols represent individual data points and bars represent group geometric mean titers. FIG. 4E shows multifunctional CD4⁺ T cell responses at various times for both the IN group and the IM group. FIG. 4F shows Th1 cytokine (IFN-γ, IL-2, TNF-α) responses evaluated in NHP circulating CD4⁺ T cells (PBMCs) on Study Day 97 and 153 (n=5 per group). Colored symbols represent individual data points and bars represent group geometric mean titers. FIGS. 4G-4J show mucosal anti-A/AW IgA and IgG titers from NHPs were analyzed in nasal wash (4G and 4I) and BAL samples (4H and 4J) collected on study days indicated on the X axis. Colored symbols represent individual data points and bars represent group geometric mean titers. Error bars represent 95% confidence intervals. Differences between groups were evaluated by Kruskal-Wallis test. Only statistically significant differences are indicated in the figure. *p<0.05; **p≤0.005; ***p<0.0005; ****p<0.00005.

FIGS. 5A-5C show head and stem neutralizing epitopes where monoclonal antibodies bind. FIG. 5A shows an in silico model of A (H5N1) A/AW/SC/2021 HA with mapping where neutralizing mAbs interact with the protein. The HA-antigen is modeled with NVX.73.2 mAb showing its interaction with the head region of A (H5N1) HA, binding (LC-light chain in blue and HC-heavy chain in purple) to head region towards the vestigial esterase (VE) domain (cyan) of HA. V_{HC} is the heavy chain variable domain and VLC is the variable light chain variable domain. The modeled 73.2 mAb represents binding of critical residues in the head region (green spheres). FIG. 5B shows HA-antigen modeled with NVX.361.4 mAb (Fab-variable heavy chain in orange and light chain in cyan) interacting with the head region of HA (in purple). The modeled 361.4 mAb represents binding of critical residues of the receptor binding site (RBS) in the head region (cyan and orange spheres) directly with HC-CDR2 and 3 (red). FIG. 5C shows competitive antibody equivalent (CAE) for mAbs CR6261 (left), NVX.73.2 (middle), and NVX.361.4 (right) in NHP sera collected on Study Days indicated on the X axes (both IM- and IN-immunized groups). Symbols represent individual animal data points and bars represent group geometric mean titers. Error bars represent 95% confidence intervals, and the horizontal dashed line represents the assay LOD. Differences among groups were evaluated by a Kruskal-Wallis test. Only statistically significant differences are indicated in the figure. *p<0.05; **p≤0.005; ***p<0.0005.

FIG. 6 shows results of a pseudovirus neutralization assay in two groups of Rhesus macaques (NHPs). The dosing regimen for the Groups 1 and 2 started as follows: a first dose at week 0 and a second dose at week 3 of am intramuscular quadrivalent seasonal influenza vaccine using four strains of recombinant HA. The dosing level was 60 μg of antigen/strain and 75 μg Matrix-M. At week 12, Group 1 (the intranasal group) received a first IN dose of 240 μg A/AW/SC/2021 H5-MNP vaccine, and a second IN dose of 60 μg of A/AW/SC/2021 H5-MNP at week 20. At week 12, Group 2 (the intramuscular group) received a first IM dose of 60 μg A/AW/SC/2021 H5-MNP vaccine, followed by a second dose of 60 μg A/AW/SC/2021 H5-MNP vaccine at week 20. The graph shows the H5N1 pseudovirus neutralization of serum extracted from the NHP subjects at week 0, 3, 5, 12, 14, 20 and 22. The neutralization was carried out in MDCK SIAT1 cells.

While the invention is amendable to various modifications and alternative forms, specifics thereof have been shown by way of example in the drawings and will be described in detail. It should be understood, however, that the intent is to cover all modifications, equivalents, and alternatives falling within the spirit and scope of the invention as defined by the appended claims.

### DETAILED DESCRIPTION OF THE INVENTION

### A. Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein the term “avian influenza virus” refers to influenza viruses found chiefly in birds but that can also infect humans or other animals. In some instances, avian influenza viruses may be transmitted or spread from one human to another. An avian influenza virus that infects humans has the potential to cause an influenza pandemic, i.e., morbidity and/or mortality in humans. A pandemic occurs when a new strain of influenza virus (a virus in which human have no natural immunity) emerges, spreading beyond individual localities, possibly around the globe, and infecting many humans at once. In embodiments, avian influenza refers to a type A influenza, strain H5N1.

As used herein, the term “about” or “approximately” when preceding a numerical value indicates the value plus or minus 10% of that value. For example, “about 100” encompasses 90 and 110. When “about” or “approximately” is used preceding a numerical range of X to Y, this includes a lower limit of 0.9X to 1.1Y. For example, about 100 to 200 encompasses 90 to 220.

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, “H5N1,” “A (H5N1)” or “(HPAI) A (H5N1)” thereof refers to highly pathogenic avian influenza vaccine formulations.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to a substance (e.g. a compound, polynucleotide, or polypeptide) or a process step, such that the substance forms the majority percent of the sample in which it is contained, or a process step is largely complete. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), microneutralization assay, and HAI titers.

As used herein the term “immunity” refers to induction of the immune system of a vertebrate wherein said induction results in the prevention, amelioration, and/or reduction of at least one symptom of an infection in said vertebrate. Immunity may also refer to a hemagglutination inhibition (HI) titer of ≥40 when VLPs of the invention have been administered to a vertebrate and said VLPs have induced an immune response against a HA of an influenza virus.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “subject” includes humans and other animals. Typically, the subject is a human. For example, the subject may be an adult, a teenager, a child (2 years to 14 years of age), an infant (1 month to 24 months), or a neonate (up to 1 month). In some aspects, the adults are seniors about 65 years or older, or about 60 years or older. In typical aspects, the adults are about 60 to about less than 75 years old, or at least about 75 years old. In some aspects, the subject is a pregnant woman or a woman intending to become pregnant. In other aspects, subject is not a human; for example, a non-human primate; for example, a baboon, a chimpanzee, a gorilla, or a macaque. In certain aspects, the subject may be a pet, such as a dog or cat.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

There is continuing interest in producing vaccines that enhance innate and adaptive immunity including by enhancement of inflammasome activation, cytokine production, and acquired induction of antigen-specific antibody and CD4⁺ and CD8⁺ T cells.

As used herein, “respiratory disease” or “respiratory disorder” means diseases or disorder of the airways and/or other structures of the lung. This includes but is not limited to chronic obstructive pulmonary disease (COPD), asthma, allergic rhinitis and sinusitis, bronchiectasis, and pulmonary hypertension. COPD refers to a group of diseases that share symptoms including one or more of airflow blockage and breathing-related problems including emphysema, chronic bronchitis, and in some cases asthma.

Subtypes of Influenza A are defined by the combination of the antigenic viral proteins hemagglutinin (H) and neuraminidase (N) in the viral envelope. For example, “H1N1” denotes an influenza A virus subtype having a type-1 hemagglutinin and a type-1 neuraminidase. Most combinations of H (types 1-16) and N (types 1-11) have been found in wild birds, while some have also been found in bats. The most common subtypes of influenza A circulating in humans are H1N1 and H3N2, although other subtypes infect humans also. Of particular to the medical community is the possibility of the highly pathogenic avian influenza A virus, A (H5N1) becoming widely infectious in humans.

In some embodiments, the present invention provides for immunogenic compositions containing nanoparticles comprising viral glycoproteins such as hemagglutinins (HAs). HAs are homotrimeric in form. In some embodiments, compositions comprise HAs purified with PS80 detergent to form HA-PS80 nanoparticles which may be formulated with saponin-based Matrix-M adjuvant to form HA trimers associated with the components of Matrix-M adjuvant, i.e. Matrix-A particles and Matrix-C particles. The immunogenic compositions of the present invention provide increased antigen density with larger APC recognition resulting in heightened immunogenic responses. The present invention also provides for methods of producing immunogenic compositions that result in broad neutralizing responses. Methods of administering immunogenic compositions to a subject are also provided.

The glycoprotein antigens, typically HA, in the nanoparticles are typically produced by recombinant expression in host cells. Typically, the glycoproteins are expressed in insect host cells using a baculovirus system. After growth of the host cells, the protein may be harvested from the host cells using detergents and purification protocols. Once the host cells have grown for 48 to 96 hours, the cells are isolated from the media and a detergent-containing solution is added to solubilize the cell membrane, releasing the protein in a detergent extract. To form detergent-core nanoparticles, the first detergent, used to extract the protein from the host cell is substantially replaced with a second detergent to arrive at the nanoparticle structure. The influenza antigen nanoparticles are produced by incubating the detergent-core nanoparticles with a Matrix-M adjuvant comprising a saponin fraction, cholesterol and a phospholipid.

As used herein, A/AW/SC/2021 refers to the A (H5N1) clade 2.3.4.4b A/American Wigeon/South Carolina/22/000345-001/2021 strain.

As used herein, HA H5-MNP refers to a novel H5-Matrix-M adjuvant nanoparticle vaccine composition. More particularly, A/AW/SC/2021 HA H5-MNP refers to a novel HPAI A (H5N1) clade 2.3.4.4b HA-Matrix-M adjuvant nanoparticle vaccine composition.

In specific embodiments, the immunogenic compositions are suitable for use in influenza seasonal and pandemic vaccines comprising: (a) influenza HA glycoprotein nanoparticle; (b) Matrix-M adjuvant; and (c) a pharmaceutically acceptable buffer.

In specific embodiments, the immunogenic compositions are suitable for intramuscular or intranasal delivery.

In specific embodiments, purified recombinant A/AW/SC/2021 HA homotrimers linked to polysorbate 80 (HA-PS80) nanoparticles are mixed with Matrix-M adjuvant, composed of saponin fractions from the Quillaja saponoria Molina tree co-formulated with cholesterol and phospholipids forming cage-like icosahedral particles with an approximate diameter of 40-50 nm. Incubation of the HA-PS80 nanoparticles with Matrix-M adjuvant results in the A/AW/SC/2021 HA homotrimers associating with a cage-like Matrix-M component nanoparticle (MNP), forming what is referred to as an H5-MNP. In particular, the C-terminus of the HA glycoprotein associates with a Matrix-M component particle, in what is believed to be a hydrophobic interaction. Such antigen/MNP structures may also be formed using different recombinant protein antigens. For example a varicella zoster virus (shingles) vaccine may include a recombinant protein antigen based on a varicella zoster virus glycoprotein, such as glycoprotein E, associated with a Matrix-M component particle. In another example, a respiratory syncytial virus (RSV) vaccine may include a recombinant protein antigen based on an RSV glycoprotein, such as RSV fusion (F) glycoprotein, associated with a Matrix-M component particle.

In specific embodiments, A (H5N1) A/AW/SC/2021 HA contains a polybasic cleave site deletion at residues 341-344.

As used herein, the terms “intranasal administration” and “nasal administration” refer to administration of an immunogenic composition described herein to the nasal cavity of a subject. In embodiments, intranasal administration is achieved using a liquid preparation (e.g., an aqueous preparation), an aerosolized preparation, or a dry powder preparation. In embodiments, the liquid preparation, aerosolized preparation, or dry powder preparation is administered via an externally propelled nasal delivery device. In embodiments, the liquid preparation, aerosolized preparation, or dry powder preparation is administered via a self-propelled (i.e., via inhalation) nasal delivery device. In embodiments, the liquid preparation, aerosolized preparation, or dry powder preparation is administered via nasal insufflation (when an immunogenic composition is blown into the nose) or nasal instillation (when an immunogenic composition is dropped into the nose). In embodiments, the liquid preparation, aerosolized preparation, or dry powder preparation is administered via a gel, cream, ointment, lotion, or paste applied to one or more nasal epithelium (e.g., olfactory epithelium or nasal respiratory epithelium). In embodiments, the immunogenic composition is applied to mucus in the nasal cavity of a subject.

As used herein, the term “non-invasive nasal delivery device” refers an instrument that is capable of delivering an immunogenic composition to the nasal cavity without piercing the epithelium of the subject. Non-limiting examples of non-invasive nasal delivery devices include propellant (e.g., a pressurized inhaler) and non-propellant (e.g., a pump-type inhaler) types of aerosol or atomizer devices, particle dispersion devices, nebulizers, and pressurized olfactory delivery devices for delivery of liquid or powder formulations.

As used herein, the term “dry powder composition” refers to a lyophilized or spray dried form of an immunogenic composition described herein. In embodiments, a dry powder composition contains less than 10%, 9%, 8%, 7%, 6%, 5%, 4%, 3%, 2%, 1%, or less residual water content.

As used herein, the term “permeability enhancer” refers to a component of an immunogenic composition formulated for intranasal administration which promotes the passage of a viral glycoprotein through the nasal epithelium.

As used herein, the terms “bedside mix,” “bedside formulations,” “bedside vaccine compositions,” “bedside vials,” “bedside vial formulations” refer to vaccine formulations that are prepared immediately prior to administration. Such vaccine formulations contain viral antigens and adjuvants that are separately stored in different containers and are administered to a subject (e.g., either administering two consecutive injections, or combining the antigens and the adjuvants into one injection prior to administration).

As used herein, the terms “co-formulation mix,” “co-formulation,” “co-formulation immunogenic compositions,” “prefilled syringes,” “pre-mix,” refer to vaccine formulations that are prepared for short to long-term storage prior to the time of administration to a subject. Such vaccine formulations contain a combination of antigens and adjuvant in the same container and prepared in advance of administration.

As used herein, the term “split-virion” refers to a virus (e.g., an influenza virus), which has a viral membrane that has been disrupted with a surfactant. Examples of surfactants are described throughout this disclosure. Split-virions do not undergo further purification, so they typically contain multiple viral proteins.

As used herein, the term “recombinant” as it refers to a protein (e.g. hemagglutinin) that is produced in a cell by transcription and translation of a nucleic acid that is introduced into a cell. The nucleic acid may be introduced via a vector or a virus encoding the nucleic acid.

As used herein, the term “whole influenza virus” refers to a virus that comprises all of its envelope, viral membrane, nucleocapsid, and genetic material. In embodiments, the whole influenza virus is inactivated.

As used herein, the term “inactivated virus” refers to a virus that has undergone treatment to substantially reduce or eliminate its virulence compared to the wild-type virus.

The term “bronchoalveolar lavage,” also referred to as “BAL” refers to a fluid sample retrieved from a patient's lungs. In embodiments, the BAL is retrieved during a bronchoscopy. During a bronchoscopy, a bronchoscope containing a solution (e.g., saline) is passed through the mouth or nose into the lungs. The solution is subsequently collected from the lungs.

The term “mucosal immunity” refers to the cellular and humoral immune response that occurs in mucosal membranes. In embodiments, the methods of the disclosure provided herein result in mucosal immunity in the respiratory system.

The term “atomization” refers to the generation of fine, inhalable droplets of a liquid. The typical dimensions of atomized droplets are in the range of several microns.

The term “aerosol” refers to a dispersion of solid or liquid particles in a gas phase. In embodiments, the gas phase is air.

### B. Intranasal Immunogenic Compositions

The nanoparticles of the present disclosure are non-naturally occurring products, the components of which do not occur together in nature. Generally, the methods disclosed herein use a detergent exchange approach wherein a first detergent is used to isolate a viral glycoprotein and then that first detergent is exchanged for a second detergent to form detergent-core nanoparticles.

### Viral Antigens

The HA glycoproteins used as influenza antigens may be from any influenza virus strain. Human influenza Type A and Type B viruses cause seasonal epidemics of disease almost every winter in the United States. Influenza Type A viruses are divided into subtypes based on two proteins on the surface of the virus: the hemagglutinin (HA) and the neuraminidase (NA).

In embodiments, the HA protein may be selected from the sub-types HI, H2, H3, H4, H5, H6, H7, H8, H9, H10, H11, H12, H13, H14, H15, H16, H17, and H18. Phylogenetically, the influenza is split into groups. For HA, Group 1 contains HI, H2, H5, H6, H8, H9, H11, H12, H13, H16, H17, and H18 and group 2 contains H3, H4, H7, H10, H14, and H15.

In embodiments, the antigen may be a full-length wild type sequence, however, the antigen may also be a variation or mutant of the wild type antigen. In certain aspects, the antigen may share identity to a disclosed antigen; for example, the percentage identity may be at least 90%, at least 95%, at least 97%, at least 98%, at least 99% or 100%. Percentage identity can be calculated using the alignment program ClustalW2, available at www.ebi.ac.uk/Tools/msa/clustalw2/. The following default parameters may be used for Pairwise alignment: Protein Weight Matrix=Gonnet; Gap Open=10; Gap Extension=0.1.

Other variants may be used. HA is a homotrimer with each monomer consisting of ˜550 amino acid residues. Each monomer of HA has been conceptually divided into three domains: the ectodomain of ˜ 515 residues constitutes the extraviral part of the molecule; a single stretch of 27 residues defines the transmembrane (TM) domain; and ˜ 10 residues constitute the cytoplasmic tail (CT) While some changes may be made to the antigens, formation of detergent core nanoparticles is favored by an intact transmembrane domain (TMD). Thus, in particular examples, a modified HA protein sequence may comprise 100% identity (i.e. is wild type) over the TM and CT domains with some flexibility in the remaining ectodomain portion, where identity may be at least 90% or at least 95%, at least 97%, at least 98%, at least 99% or 100%.

Non-limiting examples of viral glycoproteins are described below. In embodiments, the immunogenic compositions may contain HA glycoproteins from H5 flu strains. Exemplary sequences of recombinant H5 HA glycoproteins include SEQ ID Nos. 1-3, as listed below.

| SEQ. ID NO. | Strain/Sequence Information | Sequence |
|---|---|---|
| 1 | A_Mink_Spain_3691- | MENIVLLLAIVSLVKSDQICIGY |
| | 8_H5_HA | HANNSTEQVDTIMEKNVTVTHA |
| | | QDILEKTHNGKLCDLNGVKPLIL |
| | | KDCSVAGWLLGNPMCDEFIR VP |
| | | EWSYIVERANPANDLCYPGSLN |
| | | DYEELKHLLSRINHFEKILIIPKSS |
| | | WPNHETSLGVSAACPYQGAPSF |
| | | FRNVVWLIKKNDAYPTIKISYN |
| | | NTNREDLLILWGIHHSNNAEEQ |
| | | TNLYKNPTTYISVGTSTLNQRLV |
| | | PKIATRSQVNGQRGRMDFFWTI |
| | | LKPDDAIHFESNGNFIAPEYAYK |
| | | IVKKGDSTIMKSGVEYGHCNTK |
| | | CQTPVGAINSSMPFHNIHPLTIGE |
| | | CPKYVKSNKLVLATGLRNSPLR |
| | | ERGLFGAIAGFIEGGWQGMVDG |
| | | WYGYHHSNEQGSGYAADKEST |
| | | QKAMDGVTNKVNSIIDKMNTQ |
| | | FEAVGREFNNLERRIENLNKKM |
| | | EDGFLDVWTYNAELLVLMENE |
| | | RTLDFHDSNVKNLYDKVRLQLR |
| | | DNAKELGNGCFEFYHKCDNEC |
| | | MESVRNGTYDYPQYSEEARLKR |
| | | EEISGVKLESIGTYQILSIYSTAA |
| | | SSLALAIMIAGLSLWMCSNGSL |
| | | QCRICI* |
| 2 | A_Colorado_18_2022_H5_HA | MENIVLLLAIVSLVKSDQICIGY |
| | | HANNSTEQVDTIMEKNVTVTHA |
| | | QDILEKTHNGKLCDLNGVKPLIL |
| | | KDCSVAGWLLGNPMCDEFIR VP |
| | | EWSYIVERANPANDLCYPGSLN |
| | | DYEELKHMLSRINHFEKILIIPKS |
| | | SWPNHETSLGVSAACPYQGAPS |
| | | FFRNVVWLIKKNDAYPTIKISYN |
| | | NTNREDLLILWGIHHSNNAEEQ |
| | | TNLYKNPTTYISVGTSTLNQRLA |
| | | PKIATRSQVNGQRGRMDFFWTI |
| | | LKPDDAIHFESNGNFIAPEYAYK |
| | | IVKKGDSTIMKSGVEYGHCNTK |
| | | CQTPVGAINSSMPFHNIHPLTIGE |
| | | CPKYVKSNKLVLATGLRNSPLR |
| | | ERGLFGAIAGFIEGGWQGMVDG |
| | | WYGYHHSNEQGSGYAADKEST |
| | | QKAIDGVTNKVNSIIDKMNTQF |
| | | EAVGREFNNLERRIENLNKKME |
| | | DGFLDVWTYNAELLVLMENER |
| | | TLDFHDSNVKNLYDKVRLQLR |
| | | DNAKELGNGCFEFYHKCDNEC |
| | | MESVRNGTYDYSQYSEEARLKR |
| | | EEISGVKLESVGTYQILSIYSTAA |
| | | SSLALAIMMAGLSLWMCSNGSL |
| | | QCRICI* |
| 3 | A_American_Wigeon_H5_HA | MENIVLLLAIVSLVKSDQICIGY |
| | | HANNSTEQVDTIMEKNVTVTHA |
| | | QDILEKTHNGKLCDLNGVKPLIL |
| | | KDCSVAGWLLGNPMCDEFIR VP |
| | | EWSYIVERANPANDLCYPGSLN |
| | | DYEELKHMLSRINHFEKILIIPKS |
| | | SWPNHETSLGVSAACPYQGAPS |
| | | FFRNVVWLIKKNDAYPTIKISYN |
| | | NTNREDLLILWGIHHSNNAEEQ |
| | | TNLYKNPTTYISVGTSTLNQRLA |
| | | PKIATRSQVNGQRGRMDFFWTI |
| | | LKPDDAIHFESNGNFIAPEYAYK |
| | | IVKKGDSTIMKSGVEYGHCNTK |
| | | CQTPVGAINSSMPFHNIHPLTIGE |
| | | CPKYVKSNKLVLATGLRNSPLR |
| | | ERGLFGAIAGFIEGGWQGMVDG |
| | | WYGYHHSNEQGSGYAADKEST |
| | | QKAIDGVTNKVNSIIDKMNTQF |
| | | EAVGREFNNLERRIENLNKKME |
| | | DGFLDVWTYNAELLVLMENER |
| | | TLDFHDSNVKNLYDKVRLQLR |
| | | DNAKELGNGCFEFYHKCDNEC |
| | | MESVRNGTYDYPQYSEEARLKR |
| | | EEISGVKLESVGTYQILSIYSTAA |
| | | SSLALAIMMAGLSLWMCSNGSL |
| | | QCRICI* |

In embodiments, the HA glycoproteins have 90% identity, 95%, 98%, 99% or 100% identity to any one of SEQ ID NOs. 1-3. The HA glycoproteins may differ relative to any one of SEQ ID Nos 1-3 via substitution, addition, or deletion of one or more residues. The recombinant HA glycoproteins with SEQ ID NOs: 1-3 differ from their respective wild types by including a polybasic cleavage site deletion AKRRK, corresponding to wild type residues 341-344. Other substitutions that may be present include M120L, A226V, I386M, P501S, V530I, M544I, in addition to many others.

### Production of Viral Glycoproteins

Viral glycoproteins are typically produced by recombinant expression in host cells. Standard recombinant techniques may be used. In embodiments, the viral glycoproteins are expressed in insect host cells using a baculovirus system. In embodiments, from 1-50 viral glycoproteins are co-expressed in a host cell. In embodiments, the baculovirus is a cathepsin-L knock-out baculovirus, a chitinase knock-out baculovirus. Optionally, the baculovirus is a double knock-out for both cathepsin-L and chitinase. High level expression may be obtained in insect cell expression systems. Non limiting examples of insect cells include Spodoptera frugiperda (Sf) cells, e.g. Sf9, Sf21, Sf22, Trichoplusiani cells, e.g. High Five cells, and Drosophila S2 cells. In embodiments, the viral glycoproteins described herein are produced in any suitable host cell. In embodiments, the host cell is an insect cell. In embodiments, the insect cell is an Sf22 cell.

Typical transfection and cell growth methods can be used to culture the cells. Vectors, e.g., vectors comprising polynucleotides that encode fusion proteins, can be transfected into host cells according to methods well known in the art. For example, introducing nucleic acids into eukaryotic cells can be achieved by calcium phosphate co-precipitation, electroporation, microinjection, lipofection, and transfection employing polyamine transfection reagents. In one embodiment, the vector is a recombinant baculovirus.

Methods to grow host cells include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 3500 L bags.

### Purification of Glycoproteins

After growth of the host cells, the glycoprotein may be harvested from the host cells using detergents and purification protocols. In embodiments, multiple viral glycoproteins are purified simultaneously. In embodiments, host cells expressing multiple viral glycoproteins are pooled together. Once the host cells have grown for 48 to 96 hours, the cells are isolated from the media and a detergent-containing solution is added to solubilize the cell membrane, releasing the protein in a detergent extract. Triton X-100 and TERGITOL® nonylphenol ethoxylate, also known as NP-9, are each preferred detergents for extraction. The detergent may be added to a final concentration of about 0.1% to about 1.0%. For example, the concentration may be about 0.1%, about 0.2%, about 0.3%, about 0.5%, about 0.7%, about 0.8%, or about 1.0%. The range may be about 0.1% to about 0.3%. In aspects, the concentration is about 0.5%.

In other aspects, different first detergents may be used to isolate the protein from the host cell. For example, the first detergent may be Bis(polyethylene glycol bis [imidazoylcarbonyl]), nonoxynol-9, Bis(polyethylene glycol bis [imidazoyl carbonyl]), BRIJ® Polyethylene glycol dodecyl ether 35, BRIJ® Polyethylene glycol (3) cetyl ether 56, BRIJ® alcohol ethoxylate 72, BRIJ® Polyoxyl 2 stearyl ether 76, BRIJ® polyethylene glycol monoolelyl ether 92V, BRIJ® Polyoxyethylene (10) oleyl ether 97, BRIJ® Polyethylene glycol hexadecyl ether 58P, CREMOPHOR® EL Macrogolglycerol ricinoleate, Decaethyleneglycol monododecyl ether, N-Decanoyl-N-methylglucamine, n-Decyl alpha-Dglucopyranoside,Decyl beta-D-maltopyranoside, n-Dodecanoyl-N-methylglucamide, nDodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N-Nonanoyl-N-methylglucamine, N-NonanoyIN-methylglucamine, Octaethylene glycol monodecyl ether, Octaethylene glycolmonododecyl ether, Octaethylene glycol monohexadecyl ether, Octaethylene glycol monooctadecyl ether, Octaethylene glycol monotetradecyl ether, Octyl-beta-D glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, SPAN® 20 sorbitan laurate, SPAN® 40 sorbitan monopalmitate, SPAN® 60 sorbitan stearate, SPAN® 65 sorbitan tristearate, SPAN® 80 sorbitane monooleate, SPAN® 85 sorbitane trioleate, TERGITOL® secondary alcohol ethoxylate Type 15-S-12, TERGITOL® secondary alcohol ethoxylate Type 15-S-30, TERGITOL® secondary alcohol ethoxylate Type 15-S-5, TERGITOL® secondary alcohol ethoxylate Type 15-S-7, TERGITOL® secondary alcohol ethoxylate Type 15-S-9, TERGITOL® nonylphenol ethoxylate Type NP-10, TERGITOL® nonylphenol ethoxylate Type NP-4, TERGITOL® nonylphenol ethoxylate Type NP-40, TERGITOL® nonylphenol ethoxylate Type NP-7, TERGITOL® nonylphenol ethoxylate Type NP-9, TERGITOL® branched secondary alcohol ethoxylate Type TMN-10, TERGITOL® branched secondary alcohol ethoxylate Type TMN-6, TRITON™ X-100 Polyethylene glycol tert-octylphenyl ether or combinations thereof.

The nanoparticles may then be isolated from cellular debris using centrifugation. In embodiments, gradient centrifugation, such as using cesium chloride, sucrose and iodixanol, may be used. Other techniques may be used as alternatives or in addition, such as standard purification techniques including, e.g., ion exchange, affinity, and gel filtration chromatography.

For example, the first column may be an ion exchange chromatography resin, such as FRACTOGEL® EMD methacrylate based polymeric beads TMAE (EMD Millipore), the second column may be a lentil (Lens culinaris) lectin affinity resin, and the third column may be a cation exchange column such as a FRACTOGEL® EMD methacrylate based polymeric beads SO3 (EMD Millipore) resin. In other aspects, the cation exchange column may be an MMC column or a Nuvia C Prime column (Bio-Rad Laboratories, Inc). Preferably, the methods disclosed herein do not use a detergent extraction column; for example a hydrophobic interaction column. Such a column is often used to remove detergents during purification but may negatively impact the methods disclosed here.

### Formation of Detergent-Core Nanoparticles Via Detergent Exchange

To form detergent-core nanoparticles, the first detergent that was used to extract the protein from the host cell is substantially replaced with a second detergent to arrive at the nanoparticle structure. NP-9 is a preferred extraction detergent. The second detergent is typically selected from the group consisting of PS20, PS40, PS60, PS65, and PS80. Preferably, the second detergent is PS80. Typically, the detergent-core nanoparticles do not contain detectable NP-9 when measured by HPLC.

In particular aspects, detergent exchange is performed using affinity chromatography to bind glycoproteins via their carbohydrate moiety. For example, the affinity chromatography may use a legume lectin column. Legume lectins are proteins originally identified in plants and found to interact specifically and reversibly with carbohydrate residues. See, for example, Sharon and Lis, “Legume lectins—a large family of homologous proteins,” FASEB J. 1990 November; 4 (14): 3198-208; Liener, “The Lectins: Properties, Functions, and Applications in Biology and Medicine,” Elsevier, 2012. Suitable lectins include concanavalin A (con A), pea lectin, sainfoin lect, and lentil lectin. Lentil lectin is a preferred column for detergent exchange due to its binding properties. Lectin columns are commercially available; for example, Capto Lentil Lectin is available from GE Healthcare. In certain aspects, the lentil lectin column may use a recombinant lectin. At the molecular level, it is thought that the carbohydrate moieties bind to the lentil lectin, freeing the amino acids of the protein to coalesce around the detergent resulting in the formation of a detergent core providing nanoparticles having multiple copies of the antigen, e.g., glycoprotein oligomers which can be dimers, trimers, or tetramers anchored in the detergent. In embodiments, the viral glycoproteins form trimers. In embodiments, the viral glycoprotein trimers are anchored in detergent. In embodiments, each viral glycoprotein nanoparticle contains at least one trimer associated with a non-ionic core.

The detergent, when incubated with the glycoprotein to form the detergent-core nanoparticles during detergent exchange, may be present at up to about 0.1% (w/v) during early purifications steps and this amount is lowered to achieve the final nanoparticles having optimum stability. For example, the non-ionic detergent (e.g., PS80) may be about 0.005% (v/v) to about 0.1% (v/v), for example, about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the nanoparticle contains about 0.03% to about 0.05% PS80. In embodiments, the nanoparticle contains about 0.01% (v/v) PS80.

In embodiments, purified viral glycoproteins are dialyzed. In embodiments, dialysis occurs after purification. In embodiments, the viral glycoproteins are dialyzed in a solution comprising sodium phosphate, NaCl, and PS80. In embodiments, the dialysis solution comprising sodium phosphate contains between about 5 mM and about 100 mM of sodium phosphate, for example, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM sodium phosphate. In embodiments, the pH of the solution comprising sodium phosphate is about 6.5, about 6.6, about 6.7, about 6.8, about 6.9, about 7.0, about 7.1, about 7.2, about 7.3, about 7.4, or about 7.5. In embodiments, the dialysis solution comprising sodium chloride comprises about 50 mM NaCl to about 750 mM NaCl, for example, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, about 500 mM, about 510 mM, about 520 mM, about 530 mM, about 540 mM, about 550 mM, about 560 mM, about 570 mM, about 580 mM, about 590 mM, about 600 mM, about 610 mM, about 620 mM, about 630 mM, about 640 mM, about 650 mM, about 660 mM, about 670 mM, about 680 mM, about 690, about 700 mM, about 710 mM, about 720 mM, about 730 mM, about 740 mM, or about 750 mM NaCl. In embodiments, the dialysis solution comprising PS80 comprises about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the dialysis solution comprises about 25 mM sodium phosphate (pH 7.2), about 300 mM NaCl, and about 0.01% (v/v) PS80.

In embodiments, the buffer comprises 10 mM sodium phosphate, 150 mM NaCl, 100 mM arginine, 5% trehalose, and 0.03% PS80 at a pH of 7.5. In embodiments, the pharmaceutically acceptable buffer comprises 25 mM sodium phosphate, 300 mM NaCl, and 0.03% PS80 at a pH of 7.2. In embodiments, the buffer comprises 25 mM sodium phosphate, 600 mM NaCl, and 0.01% PS80 at a pH of 6.8.

Detergent exchange may be performed with proteins purified as discussed above and purified, frozen for storage, and then thawed for detergent exchange.

The stability of compositions disclosed herein may be measured in a variety of ways. In one approach, a peptide map may be prepared to determine the integrity of the antigen protein after various treatments designed to stress the nanoparticles by mimicking harsh storage conditions. Thus, a measure of stability is the relative abundance of antigen peptides in a stressed sample compared to a control sample. For example, the stability of nanoparticles containing the viral glycoproteins may be evaluated by exposing the nanoparticles to various pHs, proteases, salt, oxidizing agents, including but not limited to hydrogen peroxide, various temperatures, freeze/thaw cycles, and agitation. It is thought that the position of the glycoprotein anchored into the detergent core provides enhanced stability by reducing undesirable interactions. For example, the improved protection against protease-based degradation may be achieved through a shielding effect whereby anchoring the glycoproteins into the core at the molar ratios disclosed herein results in steric hindrance blocking protease access. Stability may also be measured by monitoring intact proteins.

### Adjuvants

In embodiments, the compositions disclosed herein may be combined with one or more adjuvants to enhance an immune response. In embodiments, the compositions are prepared without adjuvants, and are thus available to be administered as adjuvant-free compositions. Advantageously, adjuvant-free compositions disclosed herein may provide protective immune responses when administered as a single dose. Alum-free compositions that induce robust immune responses are especially useful in adults about 60 and older.

Saponins are a large family of plant-derived glycoconjugates that share a triterpene structure with a variety of glycoside side chains. Saponins have traditionally been used for making soaps based on being amphipathic. Saponins now also are used for making adjuvants based on having potent immune-stimulating properties, as taught, for example by Kensil et al., U.S. Pat. No. 5,057,540.

Saponins extracted from the bark of the South American soapbark tree Quillaja saponaria Molina contain a complex heterogeneous mixture of closely related saponins with structurally different glycosylation or acylation patterns that affect their biological activities. Quillaja saponaria Molina saponins can have a high degree of glycosyl O-acylation, a low degree of glycosyl O-acylation, or no glycosyl O-acylation in their naturally occurring forms. Saponins also can be chemically modified, for example by partial or complete deacylation or degradation.

Saponins of Quillaja saponaria Molina in particular can have potent adjuvant activity, but also can be chemically unstable, show hemolytic activity, and be associated with immediate pain at injection sites. Saponin preparations based on defined compositions of purified saponin fractions of Quillaja saponaria Molina are described, for example, by Cox et al., PCT/AU1995/000670 (WO96011711).

Incorporation of saponins of Quillaja saponaria Molina into particles comprising saponin and lipid can attenuate the chemical instability, hemolytic activity, and immediate pain when injected associated with saponins. Specific examples of particles comprising saponin and lipid include saponin based particles and saponin based antigen-presenting particles, as taught, for example, by Stertman et al., Human Vaccines & Immunotherapeutics, 2023, 19 (1): 2189885, GSK's Liposome-based Adjuvant System 01 particles, as described, for example, in Didierlaurent et al., Expert Review of Vaccines, 2017, 16 (1): 55-63, and Army Liposome Formulation Q particles, as described, for example, by Alving et al., Expert Review of Vaccines, 2020, 19 (3): 279-292.

Novavax's Matrix-M adjuvant is a formulation that is manufactured using two purified extracts of saponins of Quillaja saponaria Molina, termed saponin fraction A and saponin fraction C, which are described in detail below. To make Matrix-M adjuvant, saponin fraction A and saponin fraction C are separately mixed with cholesterol and phosphatidylcholine, in the presence of the detergent Mega-10 to form dispersions of approximately 40-50 nm-sized stable cage-like structures, designated Matrix-A and Matrix-C particles. The Mega-10 detergent is removed by diafiltration. The Matrix-A and Matrix-C particles are provided in formulations in phosphate buffer solution including 137 mM sodium chloride, 2.7 mM potassium chloride, and 9.8 mM phosphate, at pH 7.2. The Matrix-M adjuvant is obtained by mixing the Matrix-A and Matrix-C particles at a fixed weight ratio of 85:15 of Matrix-A particles to Matrix-C particles. It should be appreciated that the A (H5N1) HA glycoprotein associates with a Matrix-M component to make the H5-MNP, in other words the HA glycoprotein associates with a Matrix-A particle or a Matrix C particle of the Matrix-M adjuvant.

Considering saponins in more detail, as noted above, saponin preparations based on defined compositions of purified saponin fractions of Quillaja saponaria Molina are described, for example, by Cox et al., PCT/AU1995/000670 (WO96011711). Initially, formulation of saponins into particles was performed with a semi-purified, non-fractionated saponin extract from the bark of Quillaja saponaria Molina, termed Quil-A. This led to several of the benefits of the formulation of saponins into such particles being recognized. Ambitions to bring the technology further towards a possible product, such as an adjuvant for use in animal and human vaccines, prompted increased purification and characterization of the Quil-A extract. The tools available for separation and characterization of saponins at the time, during the mid to late 1980s, were reversed-phase high-performance liquid chromatography, also termed RP-HPLC, and thin layer chromatography, also termed TLC. The number of peaks revealed by RP-HPLC were numerous, while TLC revealed a few major bands. Saponin raw material was subjected to semi-preparative high-performance liquid chromatography, also termed HPLC, separations. By screening separated saponin materials for adjuvant activity in mice it was found that the saponins of major interest were residing in one of the major TLC bands. Further separation of the saponins by HPLC and screening for adjuvant activity and structure forming ability resulted in the definition of sub-groups of saponin fractions with different and interesting features. The major findings are summarized in TABLE 1, in which present terminology and major components are included for clarity and reference is made to what currently are termed fraction A, fraction B, and fraction C of Quillaja saponaria Molina saponins.

TABLE 1
Initial characterization of potentially useful fractions
of saponin materials obtained from Quil-A.

| | Adjuvant | | | | |
|---|---|---|---|---|---|
| | activity | Matrix | | | |
| | (non- | structure | | Mw of major | Present |
| TLC | formulated | forming | Hemolytic | known | termi- |
| band | saponin) | ability | activity | component(s) | nology |
| B1 | Poor | Poor | Low | NA | NA |
| B2 | Potent | Good | Medium | 1988 | Fraction C |
| B3 | Potent | Atypical | High | 2150 (2175 | Fraction B |
| | | structures | | and 2019) | |
| B4 | Poor/none | Good | Low | 1862 | Fraction A |

Over the years the process for fractionation of saponin raw material into fraction A and fraction C has been developed and scaled-up. For example, as described in Cox et al., PCT/AU1995/000670 (WO96011711), fractions A, B, and C can be prepared from the lipophilic fraction obtained on chromatographic separation of the crude aqueous Quillaja Saponaria Molina extract on a SEP-PAK column and elution with 70% acetonitrile in water to recover the lipophilic fraction. This lipophilic fraction can then be separated by semipreparative HPLC with elution using a gradient of from 25% to 60% acetonitrile in acidic water. Fraction A is the fraction that is eluted at approximately 39% acetonitrile. Fraction B is the fraction that is eluted at approximately 47% acetonitrile. Fraction C is the fraction that is eluted at approximately 49% acetonitrile.

As noted above, saponin preparations based on defined compositions of purified saponin fractions of Quillaja saponaria Molina are described, for example, by Cox et al., PCT/AU1995/000670 (WO96011711).

In specific embodiments, Saponin Fraction C has a purity of at least 80%, as determined by HPLC. In specific embodiments, a defined ratio of specific subcomponents within Fraction A or C are identified by mass spectrometry. In specific embodiments, Cholesterol is derived from a plant-based source (phytosterol). In specific embodiments, Phosphatidylcholine comprises POPC fatty acid chains. In specific embodiments, Mega-10 detergent is used at a concentration of 1% (w/v) during particle formation.

In specific embodiments, Matrix-A and Matrix-C particles have a mean diameter between 30 nm and 50 nm, as measured by dynamic light scattering (DLS). In specific embodiments, Matrix-A and Matrix-C particles have a polydispersity index (PDI) less than 0.2. In specific embodiments, Matrix-A and Matrix-C particles exhibit a zeta potential between −20 mV and −40 mV. In specific embodiments, the cage-like structure is confirmed by transmission electron microscopy (TEM).

In specific embodiments, a Matrix-A to Matrix-C weight ratio is between 80:20 and 90:10. In some embodiments the Matrix-A to Matrix-C ratio is around 85:15. In specific embodiments, diafiltration uses a membrane with a molecular weight cut-off (MWCO) of 10 kDa to remove Mega-10. In specific embodiments, diafiltration is performed for a minimum of 10 cycles. In specific embodiments, a phosphate buffer solution has a phosphate concentration between 5 mM and 15 mM, pH 7.2. In specific embodiments, a temperature between 20° C. and 25° C. is maintained during the mixing and diafiltration steps. In specific embodiments, the Matrix-M adjuvant is sterilized by sterile filtration using a 0.22 μm filter.

In specific embodiments, a lyoprotectant (sucrose) is included at a concentration between 5% and 10% (w/v). In specific embodiments, the final formulation pH is adjusted to between 6.8 and 7.4. In specific embodiments, the formulation is stable for at least 12 months when stored at 2-8° C. In specific embodiments, reconstitution is with sterile water.

In specific embodiments, Matrix-M adjuvant is used with an A (H5N1) protein antigen. In specific embodiments, a vaccine composition comprises Matrix-M adjuvant at a concentration between 10 μg/mL and 50 μg/mL. In specific embodiments, administration is via intramuscular (IM) or intranasal (IN) route. In specific embodiments, induction of a specific antibody isotype response (IgG1). In specific embodiments, protective efficacy is demonstrated against a specific pathogen challenge in a mouse model. In specific embodiments, the adjuvant activity displays an increase in antibody titer compared to the antigen alone. In specific embodiments, the formulation exhibits reduced reactogenicity compared to other saponin-based adjuvants.

### H5-Matrix-M Nanoparticles (H5-MNPs)

In specific embodiments, one or more A (H5N1) HA glycoproteins associate with a Matrix-M component particle, either a Matrix-A particle or a Matrix-C particle, when the detergent-core nanoparticles are incubated with Matrix-M. Thus, H5-Matrix-M nanoparticles (H5-MNPs) comprise nanoparticles having one or more HA glycoproteins associated with either a Matrix-A particle or a Matrix-C particle. The singular term “H5-Matrix-M nanoparticle” can refer to a nanoparticle based on either a Matrix-A particle or a Matrix-C particle. In a vaccine containing H5-MNPs, the ratio of H5-MNPs based on Matrix-A particles to H5-MNPs based on Matrix-C particles is dependent on the initial ratio of Matrix-A and Matrix-C component particles present in the Matrix-M (85:15 w/w) incubated with the detergent core particles to form the H5-MNPs.

In this context, the term “associate” does not mean there is a covalent bond between the HA glycoprotein and the Matrix-M particle. Rather, there is a non-covalent attraction between the two, particularly between the hydrophobic C-terminus of the HA glycoprotein and the Matrix-M component particle. Without being bound by any particular theory, it is believed that the free energy of the HA glycoprotein is reduced when its C-terminus is in close proximity to the Matrix-M component particle, and thus it is energetically favorable for the HA glycoprotein to associate with the Matrix-M component particle.

### Immunogenic Compositions

Provided herein are intranasal immunogenic compositions comprising nanoparticles comprising a viral glycoprotein. In embodiments, the immunogenic compositions are for intranasal administration. Advantageously, intranasal administration of the intranasal immunogenic compositions described herein induces mucosal immunity. The induction of mucosal immunity may result in reduction of viral transmission. In other embodiments, the immunogenic compositions are for intramuscular administration.

### C. Administration of Immunogenic Compositions

Compositions disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intramuscular, or intrasternal injection. Typically, the compositions are administered by intramuscular injection. In particular aspects, the compositions may be administered mucosally. As used herein, the term “mucosal administration” includes oral, intranasal, intravaginal, intra-rectal, and intra-tracheal.

The present disclosure provides methods of preventing influenza infection. Immunogenicity of the nanoparticle influenza vaccines disclosed herein may be determined using suitable approaches, including performing HAI assays or by measuring neutralizing antibodies. In some embodiments, the immunogenicity of the nanoparticle influenza vaccines may be compared to a commercially available influenza vaccine composition. As used herein, “commercially available influenza vaccine composition” can be any influenza vaccine compositions that are available for medical uses. For example, the commercially available influenza vaccine composition can be formulated for a trivalent or a quadrivalent injection. In some aspects, the formulation for an injection can comprise the inactivated form of the virus. In another example, the commercially available influenza vaccine composition can be formulated for a nasal spray. In some aspects, the formulation for a nasal spray can comprise attenuated or weakened forms of the virus.

The compositions may be administered to a subject in need thereof, typically a human.

Compositions may be administered on a single dose schedule or a multiple dose schedule. Multiple doses may be used in a primary immunization schedule or in a booster immunization schedule. In a multiple dose schedule the various doses may be given by the same or different routes e.g., a parenteral prime and mucosal boost, a mucosal prime and parenteral boost, etc. In some aspects, a follow-on boost dose is administered about 2 weeks, about 3 weeks, about 4 weeks, about 5 weeks, or about 6 weeks after the initial dose.

Compositions may also be used to boost an immune response boost following administration of a different type of influenza vaccine. For example, an individual may have been administered a seasonal influenza vaccine, which typically contains strains related to H1N1 and H2N3 type A influenza. The administration of the seasonal vaccine may be a single dose, or an initial dose and a follow-on dose. In embodiments, the individual may be administered with an HA glycoproteins from an H5 influenza subsequent to the seasonal influenza vaccine. In other embodiments, the individual may be provided with an H5 influenza vaccine without prior administration of a seasonal influenza vaccine.

The total amount of the H5 influenza HA in the vaccine compositions may range from of about 0.1 μg to about 100 μg. In certain embodiments, the amount of the influenza HA protein in the vaccine composition may be in the range of about 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg. Advantageously, the compositions exhibit stability up to 9 to 12 months such that the amount remaining, as measured by SRID, is a substantial percentage of the initial amount; for example, at least about 70%, at least about 75%, or at least about 80% of the initial amount.

In embodiments, the intranasal immunogenic compositions comprise one or more additional ingredient selected from the group consisting of a viscosity agent, a bulking agent, a dispersant, a filler, a carrier agent, a moisturizing agent, and a flavoring agent. Examples of each of these ingredients are described herein.

### Intranasal Administration

In embodiments, provided herein are intranasal delivery devices comprising an intranasal immunogenic composition described herein. In embodiments, the delivery device is an intranasal delivery device for the administration of liquids. Non-limiting examples of devices useful for the administration of liquid compositions include vapor devices (e.g., vapor inhalers), drop devices (e.g., catheters, single-dose droppers, multi-dose droppers, and unit-dose pipettes), mechanical spray pump devices (e.g., squeeze bottles, multi-dose metered-dose spray pumps, and single/duo-dose spray pumps), bi-directional spray pumps (e.g., breath-actuated nasal delivery devices), gas-driven spray systems/atomizers (e.g., single- or multi-dose HFA or nitrogen propellant-driven metered-dose inhalers, including traditional and circumferential velocity inhalers), and electrically powered nebulizers/atomizers (e.g., pulsation membrane nebulizers, vibrating mechanical nebulizers, and hand-held mechanical nebulizers). In embodiments, the delivery device is an intranasal device for the administration of powders or gels. Non-limiting examples of devices useful for the administration of powder compositions (e.g., lyophilized or otherwise dried intranasal immunogenic compositions) include mechanical powder sprayers (e.g., hand-actuated capsule-based powder spray devices and hand-actuated powder spray devices, hand actuated gel delivery devices), breath-actuated inhalers (e.g., single- or multi-dose nasal inhalers and capsule-based single- or multi-dose nasal inhalers), and insufflators (e.g., breath-actuated nasal delivery devices). In embodiments, the intranasal delivery device is pressurized. Additional intranasal delivery devices are described in International Publication No. 2012/105236 and U.S. Pat. No. 10,441,436, which are incorporated by reference herein in their entireties for all purposes. In embodiments, the intranasal delivery device is a prefilled syringe comprising the intranasal immunogenic composition.

In embodiments, the intranasal device is a single-use, disposable device. In another embodiment, the intranasal device is a multi- or repeat-use device. In embodiments, the single-use or multi-use device is pre-metered. In embodiments, the single-use or multi-use device is prefilled. In certain embodiments, the multi-or repeat-use device is refillable.

Methods for configuring pressurized delivery devices to achieve a particular delivery profile are known in the field. In embodiments, a pressurized nasal delivery device is configured to produce a stream, spray, puff, etc., have a particular characteristic. For example, in one embodiment, to achieve administration to the upper third of the nasal epithelium, the device is configured to produce a strong, focused stream, spray, puff, etc. In one embodiment, the strong focused spray is created by imparting circumferential and/or axial velocity onto the stream of the therapeutic composition being administered into the nose. In another embodiment, to achieve administration to a greater portion of the nasal epithelium (e.g., the entire or the lower two thirds of the nasal epithelium), the device is configured to produce a diffuse and/or weaker stream, spray, puff, etc. In some embodiments, the tip of the delivery device is configured to physically direct the stream, spray, puff, etc., to the desired intranasal location when inserted into the subject's nose. For example, a kink or bend may be introduced into the tip of the delivery device to “point” the stream, spray, puff, etc., at a targeted epithelium. In some embodiments, the delivery pattern of the device is adjustable, such that the device can be differentially configured to target the intranasal immunogenic composition to a particular epithelium, structure, or location within the nose. In certain embodiments, the pooled human immunoglobulin compositions are administered by a breath-powered technology device. In certain embodiments, the breath-powered technology provides positive pressure during administration. In certain embodiments, the positive pressure expands narrow nasal passages. In some embodiments, exhalation into the device propels the therapeutic into the nose, while at the same time closing the soft-palette, thereby reducing deposition of the therapeutic into the throat and/or lungs. In one embodiment, the breath-powered technology device administers an intranasal immunogenic composition described herein into one nostril. In one embodiment, the breath-powered technology device administers an intranasal immunogenic composition described herein into two nostrils.

Non-limiting examples of commercial intranasal delivery devices include the EQUADEL® nasal spray pump (Aptar Pharma), the Solovent dry powder device (BD Technologies), the Unidose nasal drug delivery device (Consort Medical PLC), the NasoNeb® Nasal Nebulizer (MedInvent, LLC), the VeriDoser® nasal delivery device (Mystic Pharmaceuticals), the VRx2™ nasal delivery device (Mystic Pharmaceuticals), the DirectHaler™ Nasal device (Direct-Haler A/S), the TriViar™ single-use unit-dose dry powder inhaler (Trimel Pharmaceuticals), the SinuStar™ Aerosol Delivery System (Pari USA), the Aero Pump (Aero Pump GmbH), the Fit-Lizer™ nasal delivery device (Shin Nippon Biomedical Laboratories), the LMA MAD Nasal™ device (LMA North America, Inc.), the Compleo intranasal bioadhesive gel delivery system (Trimel Pharmaceuticals), Impel's Pressurized Olfactory Delivery (POD) device (Impel Neuropharma), the ViaNase™ electronic atomizer (Kurve Technology, Inc.), the OptiNose powder delivery device (OptiNose US Inc.), and the Optinose liquid delivery device (OptiNose US Inc.). In embodiments, the intranasal delivery device is a BD Accuspray™ Nasal Spray System. In embodiments, the intranasal delivery device is an intranasal mucosal atomization device (e.g., a MAD Nasal™ atomization device or the BD Accuspray™ Nasal Spray System). When intranasal immunogenic compositions described herein are administered using an intranasal mucosal atomization device, the composition is delivered to the nasal cavity as an atomized mist.

In embodiments, the intranasal delivery device delivers the intranasal immunogenic composition to the upper respiratory tract. In embodiments, the intranasal delivery device delivers the intranasal immunogenic composition to the lower respiratory tract. In embodiments, the intranasal delivery device delivers the intranasal immunogenic composition to the lungs.

In embodiments, the disclosure provides a method for eliciting an immune response against a virus, comprising intranasally administering an immunogenic composition described herein. In embodiments, the virus is influenza or a variant thereof.

### Intramuscular Administration

Provided herein are intramuscular immunogenic compositions comprising nanoparticles comprising a viral glycoprotein. In embodiments, the immunogenic compositions are for intramuscular administration. Advantageously, intramuscular administration of the intramuscular immunogenic compositions described herein induces at least intramuscular immunity and preferably also mucosal immunity.

### D. Examples

### Example 1: Generation and Characterization of a Recombinant Full-Length a (H5N1) a/AW/SC/2021 HA Glycoprotein

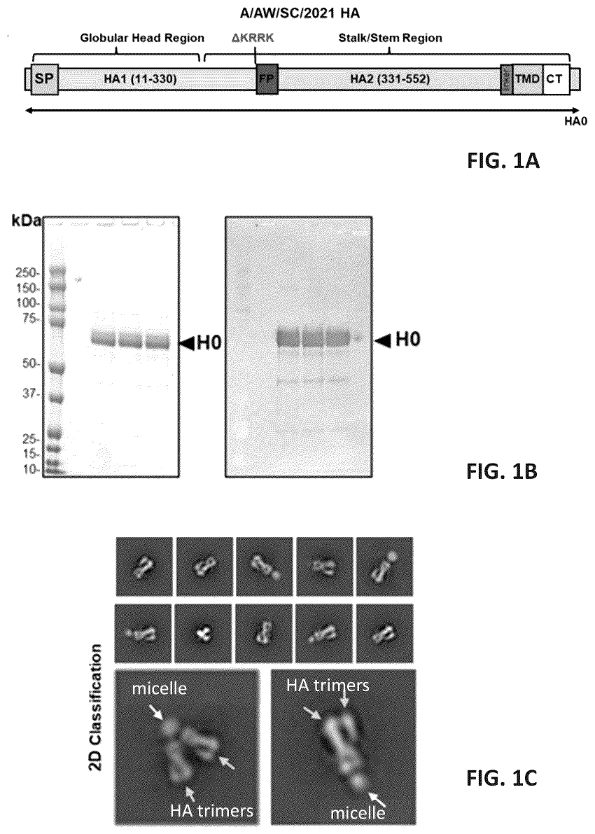

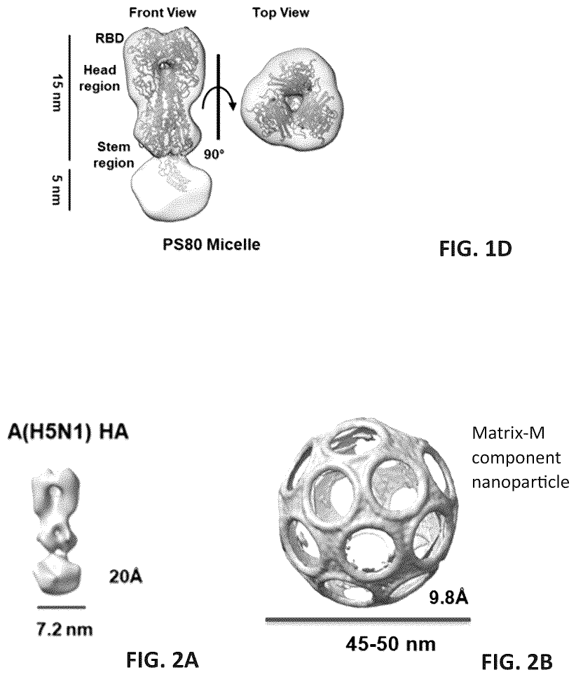

A full-length sequence (residues 1-552), including complete head and stem domains with the C-terminus transmembrane domain anchor of A (H5N1) A/AW/SC/2021 HA was designed and generated. A polybasic cleavage site deletion (AKRRK at residues 341-344) was incorporated to improve antigen stability, FIG. 1A. The antigen was cloned, produced in insect cells, and purified as described above. The purity of the protein as assessed by SDS-PAGE densitometry, see FIG. 1B, was 97.02% and the particle size was 20.95 nm in diameter as determined using dynamic light scattering (DLS), see Table 2. The thermal stability of purified protein was assessed by differential scanning calorimetry (DSC), showing a major transition with a melting temperature (Tₘ) of 52.66° C., Table 2.

TABLE 2
Particle size and thermostability of recombinant A(H5N1) HA nanoparticles

| | Dynamic light scattering | | Differential scanning calorimetry | | | |
|---|---|---|---|---|---|---|
| | Z-avg | | | | ΔH | |
| | diameter | | Tₘ (° C.) | | (kcal mol⁻¹) | |
| Influenza strain | (nm) | PDI | Tₘ₁ | Tₘ₂ | ΔH₁ | ΔH₂ |
| A(H5N1) A/AW/SC/2021 | 20.95 ± 0.09 | 0.15 ± 0.00 | 46.36 | 52.66 | 107 | 820 |
| HA | | | | | | |

ΔH, change in enthalpy;
PDI, polydispersity index;
Tₘ, melting temperature;
Z-avg, Z-average

Purified stable A/AW/SC/2021 HA antigen nanoparticles were characterized by negative staining-transmission electron microscopy (NS-TEM) and 2D class average was obtained, FIG. 1C. The NS-TEM data showed that A (H5N1) A/AW/SC/2021 HA is a homotrimer glycoprotein with dimensions of ˜138 Å (length)×15-40 Å (radius) in a prefusion state associated with a PS80 detergent micelle near its membrane anchor domain, forming a glycoprotein/detergent-core nanoparticle. 2D classification also revealed the assembly of higher-order nanoparticles, where more than one HA trimer was associated with a detergent micelle, FIG. 1C. A sequence-based in silico molecular model was generated for A/AW/SC/2021 HA and was superimposed into an electron density map reconstructed from the 2D classification, FIG. 1D, representing the intact assembly of prefusion antigen; the HAO structure was maintained for each protomer within the trimer.

Full-length HA homotrimers were observed to be linked to an extended membrane-embedded anchor along with α-helices surrounded by the detergent micelle. This is a notable feature of the HA nanoparticle, where the native-like structure of the antigen is mimicked and stabilized by PS80 detergent. This is potentially significant for immunogenicity, as accurate antigen representation enables the generation of antibodies that target various HA epitopes upon vaccination. Overall, the data suggest that the recombinant full-length A/AW/SC/2021 HA reconstituted in PS80 detergent enables a range of orientations for higher-order nanoparticle assemblies (rosettes), presenting a well-defined, flexible structure via a membrane-like anchor.

### Example 2: Structural and Biophysical Characterization of a (H5N1) a/AW/SC/2021-Matrix M Nanoparticles (H5-MNPs)

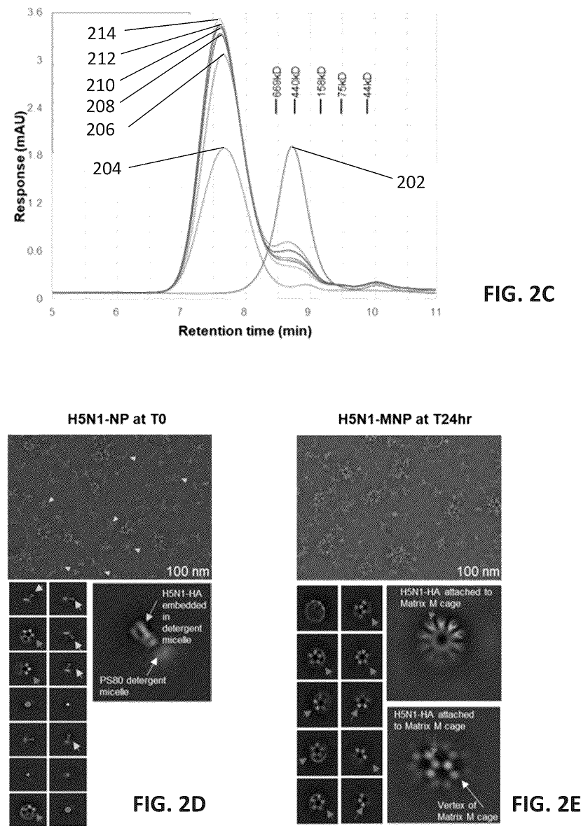

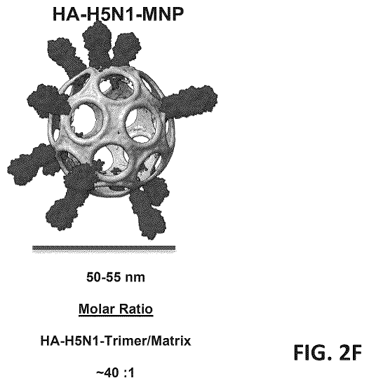

The time-dependent interactions of the influenza vaccine construct A (H5N1) A/AW/SC/2021 HA nanoparticle, see FIG. 2A, were characterized when mixed with Matrix-M adjuvant, see FIG. 2B. Purified A/AW/SC/2021 HA nanoparticles were mixed with Matrix-M at an approximately 40:1 molar ratio (corresponding to 120 μg/mL to 75 μg/mL concentration ratio by mass) and the biophysical properties were characterized using high performance-size exclusion chromatography (HP-SEC) and 2D classification by negative stain-transmission electron microscopy (NS-TEM) to investigate the kinetics of antigen-Matrix-M association. HP-SEC showed that at time 0, A (H5N1) HA and Matrix-M components eluted from the column as separate peaks corresponding to respective retention times with apexes at 8.719 min and 7.662 min. After 12 h of incubation, the eluate showed a H5-MNP peak with a shorter retention time of the apex (7.584 min) and a larger peak area, indicating that the particle size of H5-MNP became larger and approximately 95.5% of HA was bound to a Matrix component at this timepoint based on the area under the curves, see FIG. 2C. NS-TEM 2D classification identified A (H5N1) nanoparticles (HA embedded in PS80 detergent micelles) at time 0, FIG. 2D, and enabled the visualization of MNP-HA association after 24 h of incubation, FIG. 2E. HA trimers arranged themselves on MNP icosahedral cage surfaces at the vertices in a head-to-stem orientation demonstrating a direct interaction between the A/AW/SC/2021 HA transmembrane domain and the lipid: cholesterol bilayer surface vertices of Matrix-M, FIG. 2F.

Without being bound by any particular theory, it is believed that the hydrophobic nature of the HA C-terminus may drive migration of HA trimers from PS80 micelles towards the hydrophobic lipids and cholesterol of Matrix-M. Furthermore, PC80 may play a role in MNP formation related to maintaining HA trimer integrity and stability prior to formulation with Matrix-M.

The methods described here for generating H5-MNP may also be used to generate MNPs coupled to other antigens, based on the incubation of Matrix-M with detergent-core antigen nanoparticles.

### Example 3: Immunogenicity of the H5-MNPs Vaccine in Naïve Mice

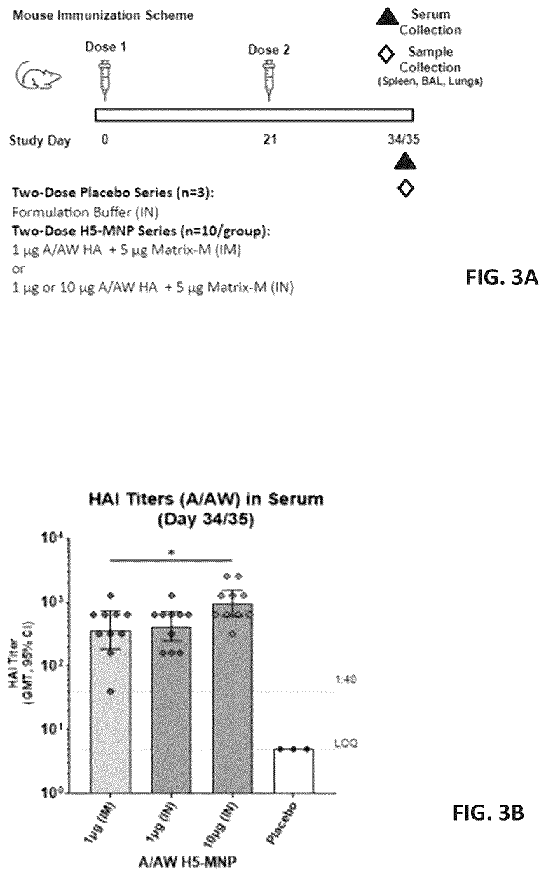

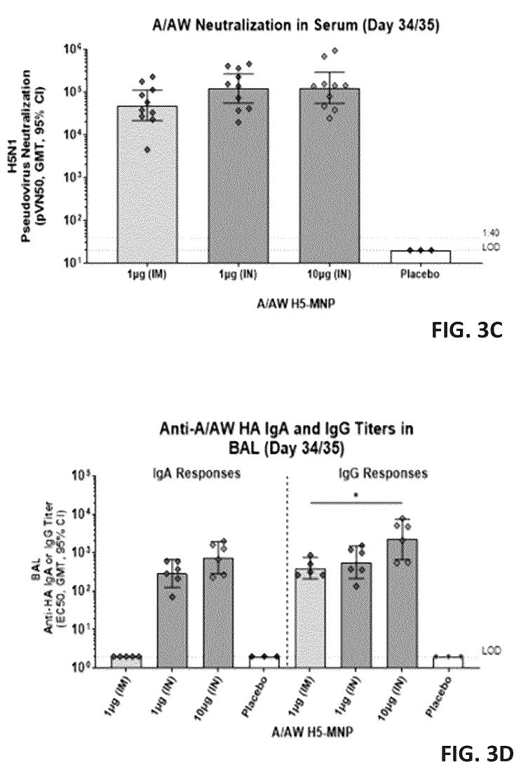

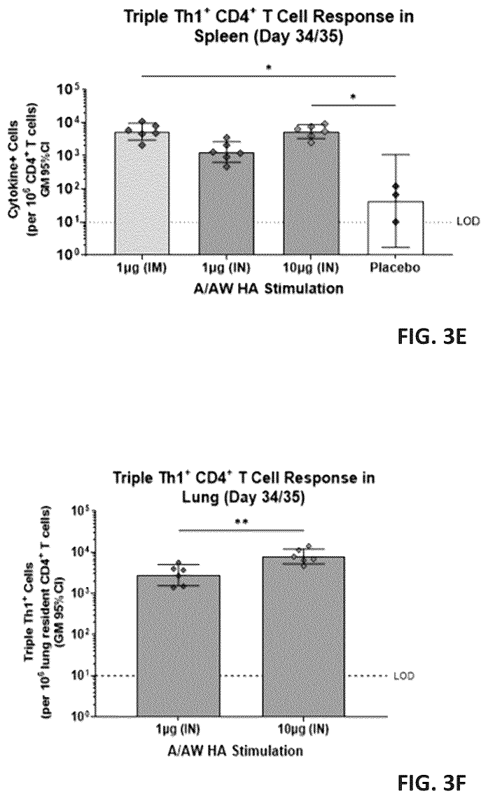

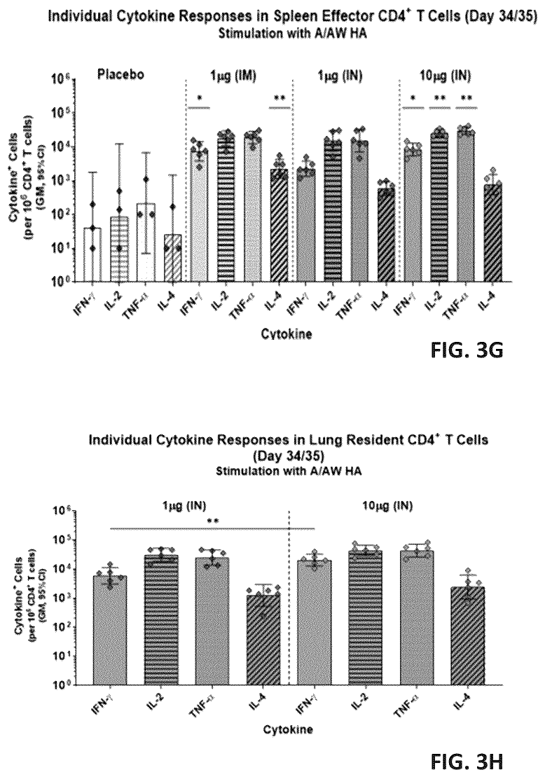

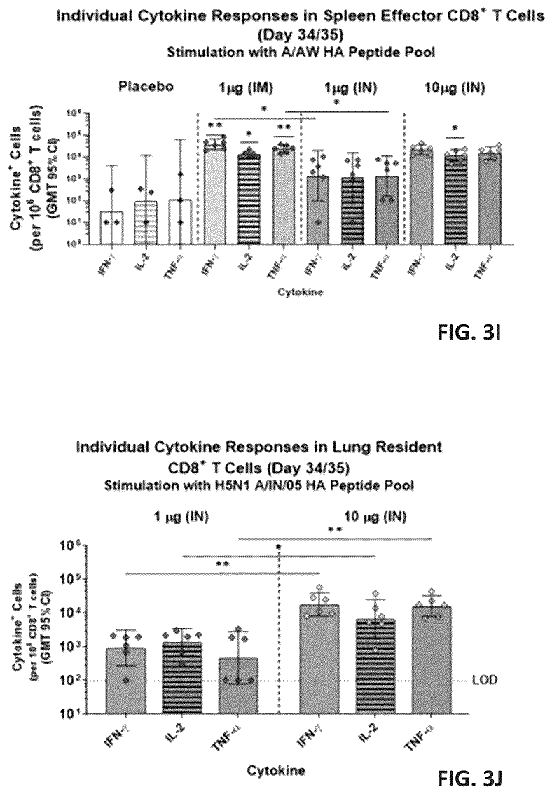

To evaluate the in vivo immunogenicity of the H5-MNP vaccine, naïve BALB/c mice were immunized with a two-dose primary series of H5-MNP administered via the intramuscular (IM) or intranasal (IN) route and evaluated antibody- and cell-mediated immune responses to vaccination. The study design is shown in FIG. 3A. Hemagglutination inhibiting (HAI) geometric mean titers (GMT) against A/AW/SC/2021 were analyzed in sera collected two weeks after the primary series; all doses and regimens of the H5-MNP vaccine elicited robust HAI antibody titers, with seroconversion observed in all immunized animals, while titers in the placebo group were undetectable, FIG. 3B. Serum HAI titers were significantly higher following immunization with 10 μg H5-MNP (IN) compared to 1 μg H5-MNP (IM) (Kruskal-Wallis test, p=0.027; Dunn's comparison, p=0.048). However, no statistically significant differences were observed between titers in the two IN dose groups, indicating that Matrix-M has an antigen-sparing effect (Dunn's comparison, p>0.05). Similarly, there were no statistically significant differences in A/AW/SC/2021 pseudovirus neutralizing titers in serum collected two weeks after the primary series across the H5-MNP treatment groups (Kruskal-Wallis test, p>0.05; Dunn's comparisons, p>0.05), FIG. 3C. All animals exhibited seroconversion, indicating that the H5-MNP regimens generated equivalent levels of functional antibodies in mice. As expected, neutralizing titers were undetectable in the placebo group.

To evaluate mucosal antibody responses to immunization, anti-A/AW/SC/2021 HA immunoglobulin A (IgA) and Immunoglobulin G (IgG) responses were determined in bronchoalveolar lavage fluid collected two weeks after the primary series, FIG. 3D. IgA titers were undetectable in the placebo-treated controls and in the H5-MNP 1 μg (IM) treatment group, indicating that IM administration of H5-MNP vaccine was ineffective at generating measurable mucosal IgA antibody responses in the lower respiratory tract of mice at this dose level. By contrast, elevated anti-A/AW/SC/2021 HA IgA responses were observed after IN administration, with both IN H5-MNP treatment groups exhibiting equivalent anti-A/AW/SC/2021 HA IgA titers (Mann-Whitney U test, p>0.05). Anti-A/AW/SC/2021 HA IgG responses in BAL were measurable in all H5-MNP treatment groups but were undetectable in the placebo-treated controls. Anti-A/AW/SC/2021 HA IgG titers in BAL were significantly higher in the 10 μg (IN) group compared to titers in the 1 μg (IM) H5-MNP treatment group (Kruskal-Wallis test, p=0.020; Dunn's comparison, p=0.028). Therefore, IN administration of the H5-MNP vaccine successfully elicited antigen-specific IgA and IgG antibody responses in the lower respiratory tract in mice, while IM administration generated IgG responses but not IgA responses in this compartment.

Regarding T cell responses, Th1 and Th2 cytokine expression was analyzed in CD4⁺ T cells from the spleen, FIG. 3E and the lung, FIG. 3F, two weeks after the primary series. In spleen effector CD4⁺ T cells, statistically significant increases in numbers of antigen-specific polyfunctional T cells (Triple Th1 cytokine positive) upon A/AW/SC/2021 HA stimulation were observed in the 1 μg (IM) and 10 μg (IN) H5-MNP-treated groups compared to placebo-treated animals (Kruskal-Wallis test, p=0.002; Dunn's comparison, p=0.015 and 0.014, respectively;

FIG. 3E). In the group administered 1 μg (IN) H5-MNP, the antigen-specific polyfunctional CD4⁺ T cell number GM in the spleen was 30.4-fold higher than the GM in the placebo group, but this difference was not statistically significant (Kruskal-Wallis test, p=0.002; Dunn's comparison, p>0.05). A similar trend was observed when examining individual Th1 cytokine expression (IFN-γ, IL-2, or TNF-α) in spleen effector cells upon stimulation with A/AW/SC/2021 HA, FIG. 3G; compared to values in the placebo group, significant increases in IFN-γ⁺ CD4⁺ T cell numbers were observed after immunization with 1 μg (IM) or 10 μg (IN) H5-MNP (Kruskal-Wallis test, p=0.002; Dunn's comparison, p=0.018 and 0.013, respectively), and significant increases in IL-2⁺ and TNF-α+ cell numbers were observed after immunization with 10 μg (IN) H5-MNP (Kruskal-Wallis test, p=0.019; Dunn's comparison, p=0.010 and Kruskal-Wallis test, p=0.008; Dunn's comparison, p=0.004, respectively). An increase of IL-4⁺ CD4⁺ T cells (Th2 cytokine) was observed after immunization with 1 μg (IM) H5-MNP (Kruskal-Wallis test, p=0.020; Dunn's comparison, p=0.002), but positive cell numbers were significantly lower than all the Th1 cytokine⁺ CD4⁺ T cell numbers, indicating a Th1-biased response. Similar to results observed for Triple Th1⁺ cytokine cell numbers, immunization with 1 μg (IN) H5-MNP did not result in statistically significant increases in individual cytokine positive CD4⁺ T cell numbers in the spleen compared to numbers in the placebo group. In spleen effector CD8⁺ T cells, the levels of all three Th1 cytokines were examined (IFN-γ, IL-2, or TNF-α) upon stimulation with the peptide pool of H5N1 A/AW HA, FIG. 3I. Compared to values in the placebo group, significant increases in IFN-γ⁺ CD8⁺ T cell numbers were observed after immunization with 1 μg (IM) H5-MNP (p=0.005), significant increases in IL-2⁺ CD8⁺ T cell numbers were observed after immunization with 1 μg (IM) or 10 μg (IN) H5-MNP (p=0.023 and 0.033, respectively), and significant increases in TNF-α⁺ cell numbers were observed after immunization with 10 μg (IM) H5-MNP (p=0.010).

Post administration, a significant number of H5N1 HA-specific resident CD4⁺ and CD8⁺ T cells were detected in the lung. Specifically, IN administration of 10 μg H5-MNP resulted in a significantly higher number of antigen-specific polyfunctional T cells than the number after administering the 1 μg H5-MNP dose (Mann-Whitney U test, p=0.0043), FIG. 3F. Individual Th1 cytokine (IFN-γ, IL-2, or TNF-α) or Th2 cytokine (IL-4) responses in lung resident CD4⁺ cells upon stimulation with A/AW/SC/2021 HA are shown in FIG. 3H. Significant increases in IFN-γ⁺ CD4⁺ T cell numbers were seen in animals immunized with a 10 μg H5-MNP dose (IN) over those immunized with a 1 μg H5-MNP dose (IN) (Mann-Whitney U test, p=0.0043). No other significant differences in individual Th1 or Th2 cytokine expression between two dosages were observed in lung resident CD4⁺ T cells. In lung resident CD8⁺ T cells, the levels of all three Th1 cytokines examined (IFN-γ, IL-2, or TNF-α) were significantly higher after IN immunization with 10 μg H5-MNP compared to responses after IN immunization with 1 μg H5-MNP upon stimulation with the peptide pool of H5N1 Indonesia HA, FIG. 3J.

In summary, the antigen- and cell-mediated immunity generating capabilities of the new A/AW/SC/2021 H5-MNP vaccine were examined in vivo in a naïve mouse model and a seasonal flu-primed NHP model. In mice, immunization with a two-dose series of an A/AW/SC/2021 H5-MNP vaccine, either IM or IN, induced robust antibody HAI titers and pseudovirus neutralization titers two weeks after vaccination. HAI assays measure the binding of head-specific antibodies targeting the receptor-binding site that inhibit red blood cell (RBC) agglutination activity. While it is desirable for novel vaccine candidates to be efficacious in generating robust HAI antibody titers, eliciting both head-specific and broader neutralizing antibody responses are advantageous for vaccine efficacy. Additionally, mice that received the H5-MNP vaccine via IN administration exhibited increased anti-A/AW/SC/2021 HA IgA titers correlating with their administration into the mucous membranes of the respiratory tract. By contrast, anti-A/AW/SC/2021 HA IgG titers were robust in all H5-MNP treatment groups suggesting a systemic antibody immune activation is present following both IM and IN administration of H5-MNP. Similar effects were observed in Th1⁺ CD4⁺ T cell activation, wherein antigen-specific lung Th1⁺ CD4⁺ T cell activation was robust following IN administration while a broader immune activation in the spleen was observed following both IM and IN administration of the H5-MNP vaccine. Overall, the H5-MNP vaccine produced neutralizing responses against A (H5N1) in naïve mice following both a standard IM injection and a less invasive IN dose that has potential, in the event of a pandemic, to be self-administered.

### Example 4: Immunogenicity of H5-MNP vaccine in non-human primates

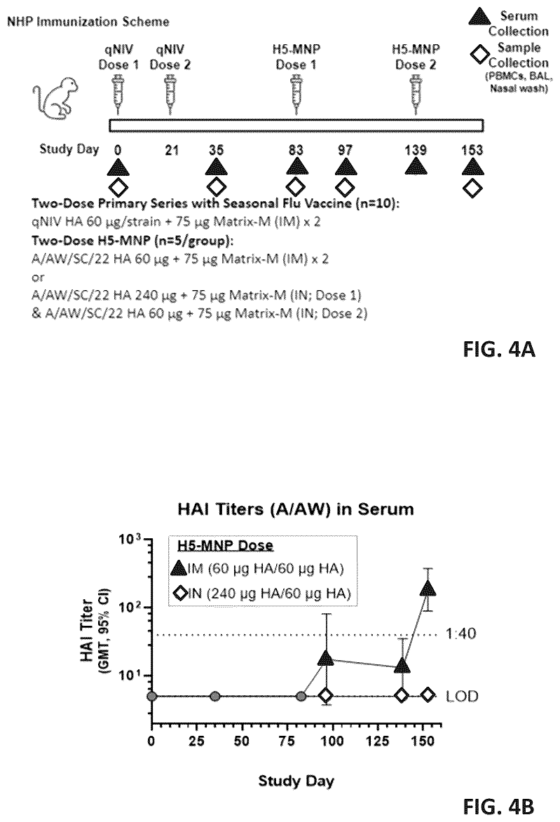

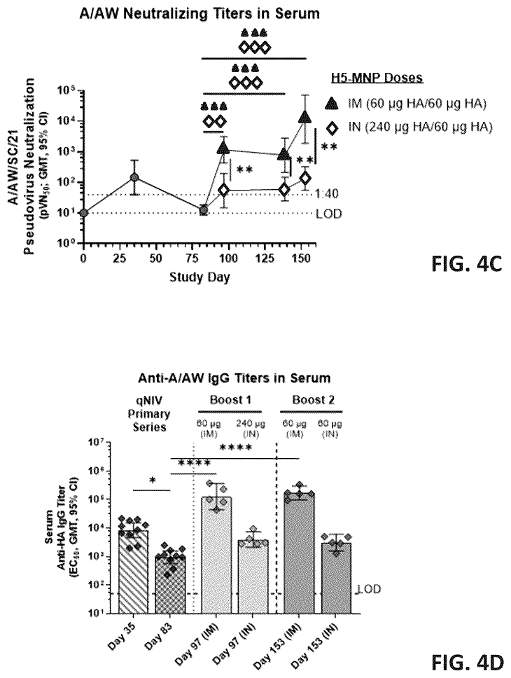

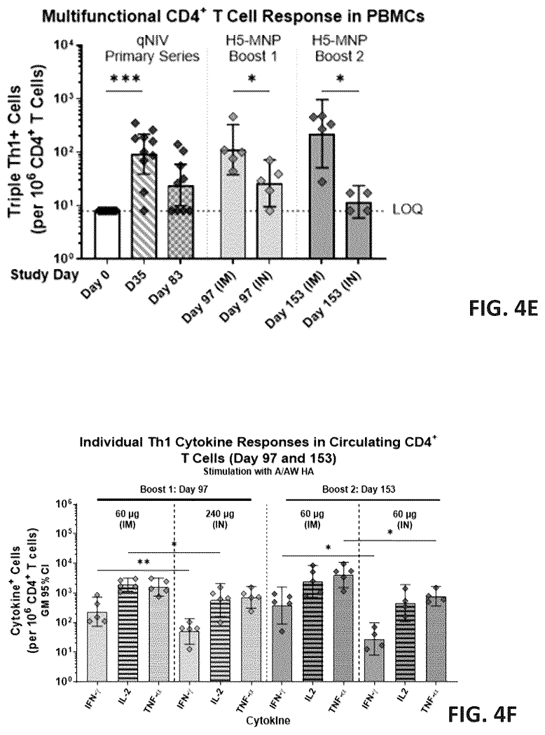

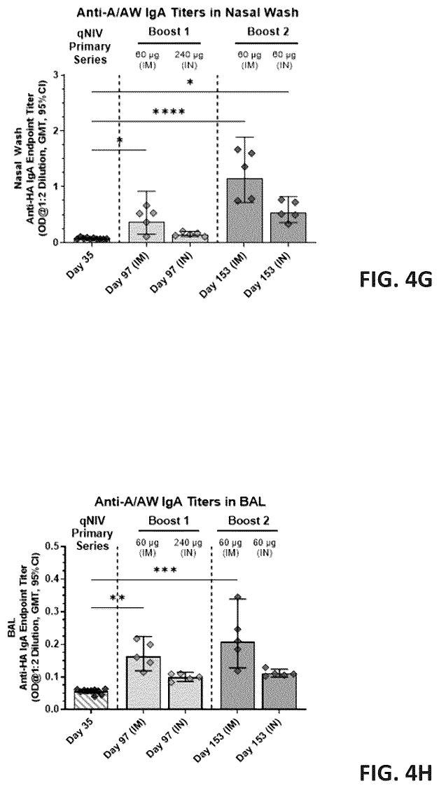

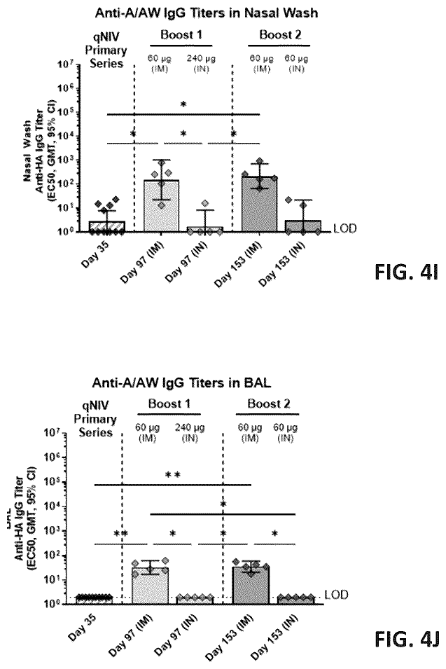

To confirm the immunogenicity of the H5-MNP vaccine in a non-human primate (NHP) model with an immune background mimicking that of the human population, an examination was made of the antibody- and cell-mediated immune responses to two IM or IN doses of the H5-MNP vaccine in Rhesus macaques primed with the Matrix-M-adjuvanted Novavax quadrivalent nanoparticle influenza vaccine (qNIV) containing HAs from 2023-2024 seasonal flu strains. The study design is shown in FIG. 4A. Immunization with two doses of qNIV resulted in undetectable HAI titers against A (H5N1) A/AW/SC/2021 in all NHPs, FIG. 4B. Administration of a single IM dose of the H5-MNP vaccine (60 μg HA with 75 μg Matrix-M adjuvant) resulted in detectable HAI (A/AW/SC/2021) titers in serum in three of five animals (two animals exhibiting seroconversion), and a second IM dose of the H5-MNP vaccine (60 μg HA with 75 μg Matrix-M adjuvant) produced detectable HAI (A/AW/SC/2021) titers in serum of all five animals (all animals exhibiting seroconversion). In contrast, two IN doses of the H5-MNP vaccine (240 μg HA or 60 μg HA, respectively, with 75 μg Matrix-M adjuvant) did not result in detectable serum HAI titers in any animal.

As another measure of functional antibody responses to immunization, neutralizing responses in sera were evaluated in NHPs throughout the study. Primary immunization with qNIV induced cross-neutralizing titers in NHPs against A/AW/SC/2021 pseudovirus with a GMT of 146, above the generally accepted seroconverting neutralizing titer of 1:40 in eight of ten animals, FIG. 4C, though these titers waned to below 1:40 in nine of ten animals after two months. A single IM or IN dose of H5-MNP vaccine significantly increased pseudovirus neutralizing titers against A/AW/SC/2021 by 92.2- and 4.3-fold to GMTs of 1160 and 54, respectively, when compared to titers before H5-MNP administration (Study Day 83) (Mann-Whitney U test, p=0.0003 and 0.008). This first H5-MNP boost resulted in 100% seroconversion after an IM dose and 80% seroconversion after an IN dose. A second IM or IN dose of H5-MNP vaccine further increased induced pseudovirus neutralizing titers against A/AW/SC/2021 to GMTs of 11698 and 134, respectively (Mann-Whitney U test, p=0.0003 and 0.0007), corresponding to 100% seroconversion after administration via either route. Significantly higher pseudovirus neutralizing titers were observed after IM administration compared to titers after IN administration (Mann-Whitney U test; Day 97, p=0.008; Day 139, p=0.008; Day 153, p=0.008). These results indicated that a single IM dose or two IN doses of H5-MNP generated potentially protective antibody responses against A/AW/SC/2021 in animals primed with seasonal flu vaccine, and that immunization via the IM route resulted in higher functional antibody titers compared to immunization via the IN route.

To measure HA-binding antibody responses, sera were also tested for anti-A/AW/SC/2021 HA IgG antibodies. The qNIV primary series resulted in measurable anti-A/AW/SC/2021 HA IgG titers on Day 35, which had significantly decreased by Day 83 (Kruskal-Wallis test, p<0.0001; Dunn's comparison, p=0.02), FIG. 4D. Animals immunized with one or two doses of IM H5-MNP, but not IN H5-MNP, showed significantly increased serum IgG titers compared to pre-boost titers on Day 83 (Kruskal-Wallis test, p<0.0001; Dunn's comparison, p<0.0001, p<0.0001, and p>0.05, respectively).

Cellular immune responses were investigated against A/AW/SC/2021 HA using peripheral blood mononuclear cells (PBMCs) collected from the NHPs primed with Matrix-M adjuvanted qNIV, followed by two IM or IN doses of H5-MNP, FIG. 4E. Measurable polyfunctional (triple Th1 cytokine positive) antigen-specific CD4⁺ T cell responses against A/AW/SC/2021 HA were elicited in NHPs primed with a two-dose primary series of 2023-2024 seasonal flu vaccine (Mann-Whitney U test, p=0.0007; Day 0 vs. Day 35). Following one H5-MNP booster dose, we observed a significantly greater CD4⁺ T cell response in the group vaccinated IM compared to the group vaccinated IN (Day 97; Mann-Whitney U test, p=0.016) and responses were also significantly higher in the IM group than in the IN group after two doses (Day 153; Mann-Whitney U test, p=0.016). Analysis of individual cytokine responses showed that IM administration of one or two doses of H5-MNP occasionally produced statistically significant increases in individual Th1 cytokine expression compared to IN administration (Day 97: Mann-Whitney U test, p=0.0079 for IFN-γ, 0.016 for IL-2; Day 153: Mann-Whitney U test, p=0.032 for IFN-γ, 0.032 for TNF-α, FIG. 4F).

Mucosal responses to H5-MNP booster doses in the upper and lower respiratory tract were also evaluated FIGS. 4G-J. Two weeks after a single IM dose of H5-MNP, anti-A/AW/SC/2021 HA IgA titers in the upper respiratory tract (nasal wash; GMT=0.371) were significantly higher than titers after the qNIV primary series (GMT=0.072; Kruskal-Wallis test, p<0.0001; Dunn's comparison, p=0.045). After the second IM H5-MNP booster dose, anti-A/AW/SC/2021 HA IgA titers in nasal wash increased further to a GMT of 1.16, corresponding to a 3.1-fold rise in GMT compared to titers after the first H5-MNP boost, though this difference was not statistically significant (Kruskal-Wallis test, p<0.0001; Dunn's comparison, p>0.05). After one IN H5-MNP boost, anti-A/AW/SC/2021 HA IgA titers in nasal wash were not significantly different than titers after the qNIV primary series (Kruskal-Wallis test, p<0.0001; Dunn's comparison, p>0.05), though the second IN H5-MNP boost did elevate titers significantly compared to titers after the primary series (GMT=0.540; Kruskal-Wallis test, p<0.0001; Dunn's comparison, p=0.011), FIG. 4G. In the lower respiratory tract (BAL), one or two IM doses of H5-MNP resulted in anti-A/AW/SC/2021 HA IgA titers of 0.147 and 0.208, respectively, which were significantly higher than titers after the qNIV primary series (Kruskal-Wallis test, p<0.0001; Dunn's comparison, p=0.011 and p=0.0011, respectively). The first and second IN booster doses of H5-MNP increased the anti-A/AW/SC/2021 HA IgA titers in BAL by less than 1.1-fold and by 2.0-fold, respectively, compared to titers after the qNIV primary series, though these increases were not statistically significant (Kruskal-Wallis test, p<0.0001; Dunn's comparison, p>0.05), FIG. 4H. Similarly, IM administration of one or two doses of H5-MNP significantly increased anti-A/AW/SC/2021 HA IgG titers in the upper and lower respiratory tracts compared to Day 35 titers (Kruskal-Wallis test, p=0.0007; Dunn's comparison, p=0.040 and p=0.017; Kruskal-Wallis test, p<0.0001; Dunn's comparison, p=0.0038 and p=0.0002, respectively), but IN administration of one or two doses did not result in any significant increase in mucosal IgG responses, and IgG responses were undetectable in BAL after IN immunization (Kruskal-Wallis test, p=0.0007; Dunn's comparison, p>0.05 and p>0.05; Kruskal-Wallis test, p<0.0001; Dunn's comparison, p>0.05 and p>0.05, respectively), FIGS. 4I, 4J,

In summary, the possibility that a seasonal flu vaccine would elicit immunity against A (H5N1) in NHPs was explored. After two doses of seasonal flu vaccine, A/AW/SC/2021 HAI antibodies were undetectable in NHPs, and though A/AW/SC/2021 neutralizing antibodies were detectable shortly after seasonal flu vaccination, antibody titers quickly waned below seroconversion levels, indicating that previous seasonal flu immunization may not be sufficient to protect individuals against an A (H5N1) influenza pandemic. While the seasonal flu vaccine does provide some antibody coverage against A (H5N1), this coverage is short-term and is unlikely to be sufficient in the case of an A (H5N1) pandemic.

In the same NHPs described above primed with qNIV (containing 2023-2024 flu strains), a single IM dose or two IN doses of an A/AW/SC/2021 H5-MNP vaccine induced significant rises in neutralizing antibody titers against the homologous A (H5N1) strain. Robust antigen-specific CD4⁺ T cell responses were also observed in PBMCs after the H5-MNP boost. Taken together, the results demonstrate the potential of H5-MNP as a pandemic vaccine in a pre-immune population. These findings highlight the potential of the A/AW/SC/2021 H5-MNP vaccine as a pandemic vaccine, although it is still unclear if additional doses/boosts would be required in adults who possess a more established immune repertoire following repeated infection and vaccinations to seasonal influenza.

Regarding mucosal responses, anti-A/AW/SC/2021 HA IgA antibody titers in the upper respiratory tract (nasal wash) of NHPs increased significantly after one IM booster dose or two IN booster doses of the H5-MNP vaccine; unexpectedly, titers were higher after the IM dose than after the IN dose. IM administration of H5-MNP also significantly increased anti-A/AW/SC/2021 HA IgA and IgG antibody titers in the lower respiratory tract (BAL), but IN administration did not significantly induce IgA antibodies in this compartment. These NHP results contrast with the findings in mice described above and also contrasts with conventional paradigms that IN immunization produces superior mucosal responses than IM immunization. Without being bound by a particular theory, it is possible that the larger nasal cavity surface area of NHPs and the higher volume of IN vaccine administered to NHPs (e.g. 500 μL for NHPs compared to 40 μL for mice) contributed to clearance from the NHP nasal mucosa before absorbing fully. If more antigen was taken in and processed after IM immunization than after IN immunization in NHPs, this may explain why IgA responses were higher in the IM group, even though IN immunization is typically known for generating stronger mucosal responses.

### Example 5: H5-MNP Vaccination Generates Neutralizing Antibody Responses Targeting HA Head and Step Epitopes

To elucidate the mechanism of how the H5-MNP vaccine elicited a neutralizing immune response in animal models, A/AW/SC/2021 HA-specific monoclonal antibodies (mAbs) were isolated from hybridomas created from immunized mice. The first antibody, mAb NVX.361.4, has heavy and light chain sequences identified respectively as SEQ. ID NO. 4 and SEQ ID NO. 5, while the second antibody, mAb NVX.73.2, has heavy and light chain sequences identified respectively by SEQ. ID NO. 6 and SEQ. ID NO. 7, as shown below.

| SEQ | Antibody | |
|---|---|---|
| ID NO. | information | Sequence |
| 4 | NVX.361.4mAb-H | MGWSYIILFLVATATGVHSQVQVQQSGSE |
| | Heavy chain | LVKPGASVRLSCKASGYNFTTYYMYWVKQ |
| | | RPGQGLEWIGEINPTNGISNFNERFKNKA |
| | | TLTVDKSSSTAYMQLSSLTSEDSAVYYCT |
| | | RASDYYGGSWFAYWGQGTLVTVSA |
| 5 | NVX.361.4mAb-L | MESQTQVFVYMLLWLSGVDGDIVMTQSQK |
| | Light chain | FMSTSVGDRVSVTCKASQNVYTNVAWYQQ |
| | | KPGQSPKALIYSASFRYNGVPDRFTGSGS |
| | | GTDFTLTISNVQSEDLADYFCQQYNNYPL |
| | | TFGAGTKLELK |
| 6 | NVX.73.2mAb-H | MAWVWTLLFLMAAAQSAQAQIQLVQSGPE |
| | Heavy chain | LKKPGETVKISCKASGYTFTNYGMNWVKQ |
| | | APGKGLKWMDWIHTYTGEPTYADDFKGRF |
| | | AFSLETSASTAYLQINNLTNEDTATYFCA |
| | | RQYGSSYGYFDYWGQGTTLTVSS |
| 7 | NVX.73.2mAb-L | METDTLLLWVLLLWVPGSTGDIVLTQSPA |
| | Light chain | SLAVSLGQRATISCRASESVDSYGKSFMH |
| | | WYQQKPGQPPKLLIYRASNLESGIPARFS |
| | | GSGSRTDFTLTINPVEADDVATYYCQQSN |
| | | EDPRTFGGGTKLEIK |

As seen in Table 3, mAb NVX.361.4 exhibited HAI activity (endpoint titer of 250 ng/ml) against A/AW/SC/2021 HA and other A (H5N1) HA proteins, and strong neutralizing activity against A/AW/SC/2021 (ID50 of 3.57 ng/mL) and other A (H5N1) HA proteins. mAb NVX.73.2 exhibited undetectable HAI activity (endpoint titer greater than 500 ng/ml) against A/AW/SC/2021 HA and other A (H5N1) HA proteins, and moderate neutralizing activity against A/AW/SC/2021 (ID50 of 42.80 ng/mL) and other A (H5N1) HA proteins.

TABLE 3
Characterization of A/AW/SC/2021 HA Monoclonal Antibodies

| | A(H5N1) mAb | |
|---|---|---|
| | NVX.361.4 | NVX.73.2 |
| A(H5N1) Virus Strains | (Head, RBS) | (Head, VE) |

Pseudovirus Neutralization
(IC₅₀, ng/mL)

| | | |
|---|---|---|
| A/AW/SC/22-000345-001/2021 HA | 3.571 | 42.8 |
| A/Colorado/18/2022 HA | 4.86 | 22.36 |
| A/Mink/Spain/3691-8 HA | 13.12 | 43.72 |

Hemagglutination Inhibition
Endpoint Titers (ng/mL)

| | | |
|---|---|---|
| A/AW/SC/22-000345-001/2021 HA | 250 | >500 |
| A/Colorado/18/2022 HA | 250 | >500 |
| A/Mink/Spain/3691-8 HA | 250 | >500 |

mAb, monoclonal antibody; RBS, receptor binding site; VE, vestigial esterase.

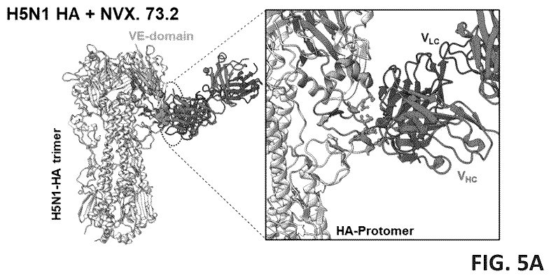

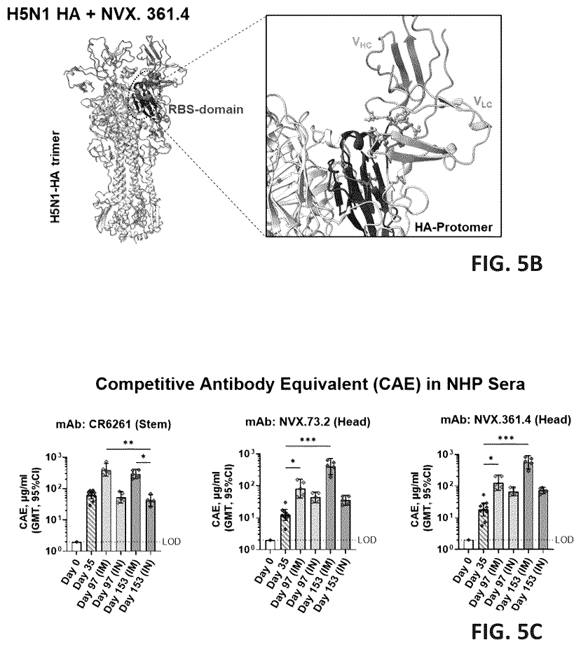

To identify the epitopes targeted by these neutralizing antibodies (NVX.73.2 and NVX.361.4), cryo-EM was utilized. Both antibodies showed binding to the globular head region of HA-A/AW/SC. Epitope mapping studies utilizing molecular modelling combined with cryoEM mapping, and NVX.73.2 was found to bind in the A/AW/SC/2021 HA head domain region, with critical residues between 60-70 (NGVKPLILKD), 90-95 (PEWSYI), and 284-290 (GVEYGHCN) (STQKAIDGVTNKVN-) making direct contact with HC-CDRs (heavy chain-complementarity determining region) and LC CDR3 (light chain-complementarity determining region 3), FIG. 5A, corresponding to the vestigial esterase (VE) subdomain based on H3 numbering. Whereas mAb NVX.361.4 mAb was revealed to bind to A/AW/SC/2021 HA in the RBS subdomain of the globular head domain above the VE subdomain with critical residues between amino acids (ETSLGV); 105-107 (GAP); 118-121 (KKND); bound to HC-CDR3 and residues 151-155 (EEQTN); 187-118 (-GQ) bound to LC-CDR2, FIG. 5B. NVX.361.4 mAb was found to bind the receptor binding site (RBS) and NVX.73.2 mAb in the VE subdomain within the HA-head domain which is conserved among H5 subtypes. The VE domain is located between the RBS in HA1 and the membrane-proximal stem region of HA2, FIG. 1A. These epitope studies confirmed that H5-MNP vaccination elicits a broadly neutralizing response by generating antibodies that bind both the RBS and VE subdomains of HA within the head region.

To confirm the in vivo generation of antibody responses against the A/AW/SC/2021 HA subdomains, sera collected from the NHP study described in Example 4 were analyzed in real-time competition binding studies, FIG. 5C. Competitive antibodies were induced in sera against all three mAbs (CR6261 targeting the HA stem region and NVX.73.2 and NVX.361.4. targeting the HA head region) in NHPs after the qNIV primary series, and a single IM dose of H5-MNP vaccine increased competitive antibody equivalent (CAE) by 6.5- to 7.0-fold against all mAbs compared to values on Day 35, which were significant for both HA head-targeting antibodies (Kruskal-Wallis test, p=0.0001; Dunn's comparison, p=0.023 and Kruskal-Wallis test, p<0.0001; Dunn's comparison, p=0.013, respectively). A similar trend was observed after the second IM dose, where CAEs for all mAbs remained 31.1- to 32.3-fold higher compared to values on Day 35, with significantly higher CAEs against HA head antibodies (Kruskal-Wallis test, p=0.0001; Dunn's comparison, p=0.0001 and Kruskal-Wallis test, p<0.0001; Dunn's comparison, p=0.0001, respectively). IN administration of H5-MNP resulted in less robust CAEs against the HA stem-targeting mAb: after the first IN boost, the level of antibodies competitive against HA stem mAb CR6261 decreased 1.5-fold compared to levels after the primary series, and after the second IN boost, CAE against this mAb decreased another 1.3-fold, though these decreases were not statistically significant (Kruskal-Wallis test, p=0.0004; Dunn's comparison, p>0.05). For the two antibodies competitive against the HA head, after the first IN booster dose, CAE against mAb NVX.73.2 increased 2.8-fold and CAE against mAb NVX.361.4 increased 10.0-fold, though neither of these differences were statistically significant compared to CAE on Day 35 (Kruskal-Wallis test, p=0.0001; Dunn's comparison, p>0.05 and Kruskal-Wallis test, p<0.0001; Dunn's comparison, p>0.05, respectively). These CAE values did not change significantly after the second IN booster dose (Kruskal-Wallis test, p=0.0001; Dunn's comparison, p>0.05 and Kruskal-Wallis test, p<0.0001; Dunn's comparison, p>0.05, respectively). Overall, the levels of antibodies competing against all three mAbs were 7.0- to 11.4-fold higher in animals administered two IM doses of H5-MNP vaccine compared to levels after two IN doses, though the only statistically significant difference was for anti-HA stem mAb CR6261 (Kruskal-Wallis test, p=0.0004; Dunn's comparison, p=0.026).

The A (H5N1) HA prefusion trimers anchored to Matrix-M adjuvant offer several advantages over compositions lacking such anchoring. Without being bound by any particular theory, is it understood that the antigen density and load of the vaccine are high, which allows for more robust activation of antigen-presenting cells (APCs). Furthermore, the H5-MNPs ensure antigen and adjuvant are co-delivered into endosomes so that, following the disruption of endosomal membranes by the release of saponins in Matrix-M, some of the antigen may be released into the cytoplasm of APCs. This may help the antigen avoid proteolysis, interact with MHC I, and induce CD8⁺ T cell responses.

### Example 6: H5-MNP Vaccination Generates Boosts Pseudovirus Neutralization

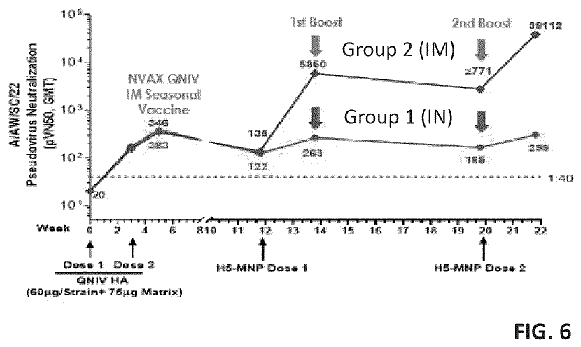

FIG. 6 shows results of a pseudovirus neutralization assay performed in two groups of Rhesus macaques (NHPs), with Group 1 receiving intranasal H5-MNP dosing and Group 2 receiving intramuscular H5-MNP dosing. The dosing regimen for both Groups 1 and 2 started as follows: a first dose at week 0 and a second dose at week 3 of am intramuscular quadrivalent seasonal influenza vaccine using four strains of recombinant HA. The dosing level was 60 μg of antigen/strain and 75 μg Matrix-M. At week 12, Group 1 received a first IN dose of 240 μg A/AW/SC/2021 H5-MNP vaccine, and a second IN dose of 60 μg of A/AW/SC/2021 H5-MNP at week 20. At week 12, Group 2 received a first IM dose of 60 μg A/AW/SC/2021 H5-MNP vaccine, followed by a second dose of 60 μg A/AW/SC/2021 H5-MNP vaccine at week 20. The graph shows the H5N1 pseudovirus neutralization of serum extracted from the NHP subjects at weeks 0, 3, 5, 12, 14, 20 and 22. The pseudovirus used was an A/AW/SC/22 H5N1 pseudovirus and neutralization was carried out in MDCK SIAT1 cells. As can be seen in the graph, there was an initial neutralization response in both Groups from the quadrivalent seasonal influenza vaccine, peaking at about week 5, and declining to week 12 when each Group received the first H5-MNP dose. Group 1 saw an initial recovery starting at week 12, peaking about week 14, followed by a gradual decline to week 20 when the second H5-MNP dose was administered, resulting in a second recovery. Group 2 also saw an initial recovery starting at week 12 and peaking at week 14, but this recovery was significantly stronger than for Group 1, suggesting that intramuscular administration of the H5-MNP was more successful at providing systemic immunity to H5N1 that intranasal administration. The neutralization values following administration of the H5-MNP vaccine are summarized in Table 4.

TABLE 4
A/AW/SC/21 pseudovirus neutralization
following administration of H5-MNP

| H5N1 | | | | |
|---|---|---|---|---|
| (A/AW/SC/21) | | | | |
| HA-Matrix | H5-MNP Dose 1 | | H5-MNP Dose 2 | |
| MNP | GMT | % MN > 1:40 | GMT | % MN > 1:40 |
| Group 1 (IN) | 263 | 80% | 299 | 100% |
| Group 2 (IM) | 5,860 | 100% | 38,112 | 100% |

### E. Applicability as a Human Vaccine

Example 5 discusses whether a seasonal influenza vaccine would elicit immunity against A (H5N1) in NHPs. After two doses of seasonal flu vaccine, A/AW/SC/2021 HAI antibodies were undetectable in NHPs, and though A/AW/SC/2021 neutralizing antibodies were detectable shortly after seasonal flu vaccination, antibody titers quickly waned below seroconversion levels, indicating that previous seasonal influenza immunization may not be sufficient to protect individuals against an A (H5N1) influenza pandemic. The immune responses and protection against H5N1 challenge were attributed to the H1N1 component of their seasonal influenza vaccine, possibly due to shared epitopes between H5 and H1, which may explain the result in NHPs that qNIV immunization elicited H5N1-neutralizing antibodies. These studies suggest that, while the seasonal influenza vaccine does provide short-lived, limited antibody responses against A (H5N1), this coverage is unlikely to be sufficient in the event of an A (H5N1) pandemic.

In the same NHPs described above primed with quadrivalent seasonal flu vaccine (“qNIV,” containing 2023-2024 influenza strains), a single IM or IN dose of an A/AW/SC/2021 H5-MNP vaccine increased neutralizing antibody titers against the homologous A (H5N1) strain, with geometric mean titers (GMTs) above the 1:40 threshold. A single IM or IN dose was sufficient to seroconvert 100% or 80% of qNIV-primed NHPs, respectively, and two IN doses were required to induce neutralizing responses in 100% of qNIV-primed NHPs. Robust antigen-specific CD4⁺ T cell responses were also observed in PBMCs after the H5-MNP boost. Taken together, the results demonstrate the potential application of H5-MNP as a pandemic vaccine in a pre-immune population. This highlights the potential use of the A/AW/SC/2021 H5-MNP vaccine as an effective pandemic vaccine. Two IN doses may be considered as an at-home self-administered booster in seasonal influenza-primed individuals during a pandemic and a single IN or IM H5-MNP vaccine dose could be valuable for pandemic preparedness.

As with all influenza HAs, the major surface glycoprotein H5 HA is responsible for most elicited humoral immune responses, but the immunodominant head domain mutates rapidly. Thus, vaccines ideally elicit immunity against more conserved epitopes on the HA protein to increase the likelihood that the vaccine will protect against homologous and drifted A (H5N1) strains. The H5-MNP vaccine described here is expected to elicit cross-neutralizing responses against other A (H5N1) strains because NHPs primed with a high dose, adjuvanted qNIV vaccine followed by one or two IM doses of the H5-MNP vaccine boosted the levels of neutralizing antibodies that target highly conserved critical residues within HA RBS, VE subdomain, and stem, and the mAbs isolated from H5-MNP vaccinated mice showed cross-neutralizing activity against two heterologous influenza A (H5N1) strains.

Possible additional advantages of an H5-MNP vaccine compared to other A (H5N1) vaccines include favorable temperature stability for storage and transport (2-8° C.), as opposed to the cold storage required for mRNA-based vaccines. the.

Various modifications, equivalent processes, as well as numerous structures to which the present invention may be applicable will be readily apparent to those of skill in the art to which the present invention is directed upon review of the present specification. The claims are intended to cover such modifications.

As noted above, the present invention is applicable to immunogenic compositions for intramuscular or intranasal delivery, in particular to immunogenic compositions containing nanoparticles comprising Avian influenza viral glycoproteins associated with Matrix-M adjuvant components. Accordingly, the present invention should not be considered limited to the particular examples described above, but rather should be understood to cover all aspects of the invention as fairly set out in the attached claims.

### EMBODIMENTS

1. An influenza vaccine nanoparticle comprising:
   a recombinant avian influenza hemagglutinin (HA) glycoprotein, wherein the HA glycoprotein is derived from Type A influenza, subtype A (H5N1); and a Matrix-M adjuvant.
2. The vaccine nanoparticle of embodiment 1, wherein the HA glycoprotein has a hydrophobic C-terminus associated with a component of the Matrix-M adjuvant.
3. The vaccine nanoparticle of embodiment 2, wherein the component of the Matrix-M adjuvant comprises one of a Matrix-A particle and a Matrix-C particle, and the hydrophobic C-terminus of the HA glycoprotein is associated with the one of the Matrix-A particle or the Matrix-C particle.
4. The vaccine nanoparticle of any of embodiments 1-3, wherein the HA glycoprotein has a sequence with at least 90% identity to, at least 95% identity to, at least 97% identity to, at least 98% identity to, at least 99% identity to, or 100% identity to any of SEQ ID NOs. 1-3.
5. The vaccine nanoparticle of embodiment 4, wherein the HA glycoprotein comprises a cytoplasmic tail (CT), a transmembrane (TM) domain and an ectodomain region and, for any one of SEQ ID NOS. 1-3, the CT and TM domain comprise 100% identity respectively to CT and TM domains of the one of SEQ ID NOS. 1-3 while the ectodomain region has a sequence with at least 90% identity to, at least 95% identity to, at least 97% identity to, at least 98% identity to, at least 99% identity to, or 100% identity to an ectodomain region of the one of SEQ ID NOS. 1-3.
6. The vaccine nanoparticle of any of embodiment 1-5, having an HA: Matrix-M adjuvant mass ratio of about 15:50 or about 15:75 or about 60:50 or about 60:75 or about 180:50 or about 180:75.
7. An immunogenic influenza composition comprising the vaccine nanoparticle of any of embodiments 1-6, and a pharmaceutically acceptable buffer.
8. The immunogenic influenza composition of embodiment 7, wherein the Matrix-M adjuvant is present at about 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.
9. The immunogenic influenza composition of any of embodiments 7-8, wherein the HA glycoprotein is present at 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.
10. A method of preparing a composition of the vaccine nanoparticle of any of embodiments 1-6, comprising extracting the HA glycoprotein from a host cell using a first detergent, exchanging the first detergent with a second detergent to form a purified detergent-core nanoparticle comprising the HA glycoprotein and the second detergent, and incubating Matrix-M adjuvant with the purified detergent-core nanoparticle to form a Matrix nanoparticle (MNP) comprising a Matrix-M component and the HA glycoprotein.
11. The method of embodiment 10, wherein the Matrix-M adjuvant and the purified detergent-core nanoparticle are incubated with an initial respective molar ratio of 1:40.
12. The method of any of embodiments 10-11, wherein the Matrix-M adjuvant is incubated with the purified detergent-core nanoparticle for a time of at least four hours.
13. The method of any of embodiments 10-12, further comprising expressing the HA glycoprotein in the host cell using a baculovirus.
14. The method of any of embodiments 10-13, wherein the host cell is a Spodoptera frugiperda (Sf) Sf9 cell or Sf22 cell.
15. The method of any of embodiments 10-14, wherein the second detergent is PS80.
16. A method of stimulating an immune response against influenza comprising administering to a subject the immunogenic influenza composition of embodiment 7.
17. The method of embodiment 16, wherein administering the immunogenic influenza composition is performed after the subject has been administered a seasonal influenza vaccine.
18. The method of any of embodiments 16-17, wherein the composition is administered intramuscularly.
19. The method of any of embodiments 16-17, wherein the composition is administered intranasally.
20. The method of any of embodiments 16-19, wherein administering the immunogenic influenza composition comprises administering about 60 μg of HA glycoprotein.
21. The method of any of embodiments 16-20, wherein administering the immunogenic influenza composition comprises administering about 75 μg of Matrix-M adjuvant.
22. A prefilled syringe containing the immunogenic influenza composition of embodiment 7.
23. A prefilled intranasal delivery device containing the immunogenic influenza composition of embodiment 7.
24. A vaccine composition, comprising
   (i) a recombinant glycoprotein antigen, the recombinant glycoprotein antigen having an N-terminal and a C-terminal;
   (ii) a Matrix-M adjuvant, wherein the C-terminal of the glycoprotein antigen is hydrophobic and is associated with a component of the Matrix-M adjuvant; and
   (iii) a pharmaceutically acceptable carrier.
25. The vaccine composition of embodiment 24, wherein the component of the Matrix-M adjuvant comprises is a Matrix-A particle or a Matrix-C particle, and the C-terminus of the recombinant glycoprotein antigen is associated with the Matrix-A particle or the Matrix-C particle
26. The vaccine composition of any of embodiments 24-25, wherein the recombinant glycoprotein is derived from an influenza hemagglutinin.
27. The vaccine composition of embodiment 26, wherein the influenza hemagglutinin is derived from type A influenza, subtype H5.
28. The vaccine composition of any of embodiments 24-27, wherein the Matrix-M adjuvant comprises Matrix-A particles and Matrix-C particles mixed at a weight ratio of 85:15.
29. The immunogenic influenza composition of any of embodiments 24-28, wherein the Matrix-M adjuvant is present at about 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.
30. The immunogenic influenza composition of any embodiments 24-29, wherein the recombinant antigen is present at 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.

What is claimed is:

1. An influenza vaccine nanoparticle comprising:
a recombinant avian influenza hemagglutinin (HA) glycoprotein, wherein the HA glycoprotein is derived from Type A influenza, subtype A (H5N1); and
a Matrix-M adjuvant.

2. The vaccine nanoparticle of claim 1, wherein the HA glycoprotein has a hydrophobic C-terminus associated with a component of the Matrix-M adjuvant.

3. The vaccine nanoparticle of claim 2, wherein the component of the Matrix-M adjuvant comprises one of a Matrix-A particle and a Matrix-C particle, and the hydrophobic C-terminus of the HA glycoprotein is associated with the one of the Matrix-A particle and the Matrix-C particle.

4. The vaccine nanoparticle of claim 1, wherein the HA glycoprotein has a sequence with at least 90% identity to, at least 95% identity to, at least 97% identity to, at least 98% identity to, at least 99% identity to, or 100% identity to any of SEQ ID Nos. 1-3.

5. The vaccine nanoparticle of claim 4, wherein the HA glycoprotein comprises a cytoplasmic tail (CT), a transmembrane (TM) domain and an ectodomain region and, for any one of SEQ ID NOS. 1-3, the CT and TM domain comprise 100% identity respectively to CT and TM domains of the one of SEQ ID NOS. 1-3 while the ectodomain region has a sequence with at least 90% identity to, at least 95% identity to, at least 97% identity to, at least 98% identity to, at least 99% identity to, or 100% identity to an ectodomain region of the one of SEQ ID NOS. 1-3.

6. The vaccine nanoparticle of claim 1, having an HA: Matrix-M adjuvant mass ratio of about 15:50 or about 15:75 or about 60:50 or about 60:75 or about 180:50 or about 180:75.

7. An immunogenic influenza composition comprising the vaccine nanoparticle of claim 1, and a pharmaceutically acceptable buffer.

8. The immunogenic influenza composition of claim 7, wherein the Matrix-M adjuvant is present at about 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.

9. The immunogenic influenza composition of claim 7, wherein the HA glycoprotein is present at 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.

10. A method of preparing the vaccine nanoparticle of claim 1, comprising extracting the HA glycoprotein from a host cell using a first detergent, exchanging the first detergent with a second detergent to form a purified detergent-core nanoparticle comprising the HA glycoprotein and the second detergent, and incubating Matrix-M adjuvant with the purified detergent-core nanoparticle to form a Matrix nanoparticle (MNP) comprising a Matrix-M component and the HA glycoprotein.

11. The method of claim 10, wherein the Matrix-M adjuvant and the purified detergent-core nanoparticle are incubated with an initial respective molar ratio of 1:40.

12. The method of claim 10, wherein the Matrix-M adjuvant is incubated with the purified detergent-core nanoparticle for a time of at least four hours.

13. The method of claim 10, further comprising expressing the HA glycoprotein in the host cell using a baculovirus.

14. The method of claim 10, wherein the host cell is a Spodoptera frugiperda (Sf) Sf9 cell or Sf22 cell.

15. The method of claim 10, wherein the second detergent is PS80.

16. A method of stimulating an immune response against influenza comprising administering to a subject the immunogenic influenza composition of claim 7.

17. The method of claim 16, wherein administering the immunogenic influenza composition is performed after the subject has been administered a seasonal influenza vaccine.

18. The method of claim 16, wherein the composition is administered intramuscularly.

19. The method of claim 16, wherein the composition is administered intranasally.

20. The method of claim 16, wherein administering the immunogenic influenza composition comprises administering about 60 μg of HA glycoprotein.

21. The method of claim 16, wherein administering the immunogenic influenza composition comprises administering about 75 μg of Matrix-M adjuvant.

22. A prefilled syringe containing the immunogenic influenza composition of claim 7.

23. A prefilled intranasal delivery device containing the immunogenic influenza composition of claim 7.

24. A vaccine composition, comprising
(i) a recombinant glycoprotein antigen, the recombinant glycoprotein antigen having a C-terminus;
(ii) a Matrix-M adjuvant, wherein the C-terminus of the glycoprotein antigen is hydrophobic and is associated with a component of the Matrix-M adjuvant; and
(iii) a pharmaceutically acceptable carrier.

25. The vaccine composition of claim 24, wherein the component of the Matrix-M adjuvant comprises a Matrix-A particle and a Matrix-C particle, and the C-terminus of the recombinant glycoprotein antigen is associated with the Matrix-A particle or the Matrix-C particle.

26. The vaccine composition of claim 24, wherein the recombinant glycoprotein is derived from an influenza hemagglutinin.

27. The vaccine composition of claim 24, wherein the influenza hemagglutinin is derived from type A influenza, subtype H5.

28. The vaccine composition of claim 24, wherein the Matrix-M adjuvant comprises Matrix-A particles and Matrix-C particles mixed at a weight ratio of 85:15.

29. The immunogenic influenza composition of claim 24, wherein the Matrix-M adjuvant is present at about 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.

30. The immunogenic influenza composition of claim 24, wherein the recombinant antigen is present at 0.1 μg to about 80 μg, about 0.1 to about 60 μg, about 5 μg to about 75 μg, about 10 μg to about 65 μg, about 20 μg to about 60 μg, about 30 μg to about 55 μg, about 35 μg to about 50 μg, or about 15 μg to about 60 μg.


SEQUENCE LISTING (7 sequences)

SEQ ID NO: 1 - protein, 563 aa, synthetic construct
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHLLSRINHFEKILIIPKSSWPNHETSLGVSAACPYQGAPSFFRNVVWLIKKNDAYPTIKISYNNTNREDLLILWGIHHSNNAEEQTNLYKNPTTYISVGTSTLNQRLVPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLRERGLFGAIAGFIEGGWQGMVDGWYGYHHSNEQGSGYAADKESTQKAMDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTAASSLALAIMIAGLSLWMCSNGSLQCRICI

SEQ ID NO: 2 - protein, 563 aa, synthetic construct
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHMLSRINHFEKILIIPKSSWPNHETSLGVSAACPYQGAPSFFRNVVWLIKKNDAYPTIKISYNNTNREDLLILWGIHHSNNAEEQTNLYKNPTTYISVGTSTLNQRLAPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLRERGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYSQYSEEARLKREEISGVKLESVGTYQILSIYSTAASSLALAIMMAGLSLWMCSNGSLQCRICI

SEQ ID NO: 3 - protein, 563 aa, synthetic construct
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHMLSRINHFEKILIIPKSSWPNHETSLGVSAACPYQGAPSFFRNVVWLIKKNDAYPTIKISYNNTNREDLLILWGIHHSNNAEEQTNLYKNPTTYISVGTSTLNQRLAPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLRERGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESVGTYQILSIYSTAASSLALAIMMAGLSLWMCSNGSLQCRICI

SEQ ID NO: 4 - protein, 140 aa, synthetic construct
MGWSYIILFLVATATGVHSQVQVQQSGSELVKPGASVRLSCKASGYNFTTYYMYWVKQRPGQGLEWIGEINPTNGISNFNERFKNKATLTVDKSSSTAYMQLSSLTSEDSAVYYCTRASDYYGGSWFAYWGQGTLVTVSA

SEQ ID NO: 5 - protein, 127 aa, synthetic construct
MESQTQVFVYMLLWLSGVDGDIVMTQSQKFMSTSVGDRVSVTCKASQNVYTNVAWYQQKPGQSPKALIYSASFRYNGVPDRFTGSGSGTDFTLTISNVQSEDLADYFCQQYNNYPLTFGAGTKLELK

SEQ ID NO: 6 - protein, 139 aa, synthetic construct
MAWVWTLLFLMAAAQSAQAQIQLVQSGPELKKPGETVKISCKASGYTFTNYGMNWVKQAPGKGLKWMDWIHTYTGEPTYADDFKGRFAFSLETSASTAYLQINNLTNEDTATYFCARQYGSSYGYFDYWGQGTTLTVSS

SEQ ID NO: 7 - protein, 131 aa, synthetic construct
METDTLLLWVLLLWVPGSTGDIVLTQSPASLAVSLGQRATISCRASESVDSYGKSFMHWYQQKPGQPPKLLIYRASNLESGIPARFSGSGSRTDFTLTINPVEADDVATYYCQQSNEDPRTFGGGTKLEIK